(19) United States
(12) Patent Application Publication
(10) Pub. No.: US 2025/0331913 A1
(43) Pub. Date: Oct. 30, 2025

(54) MANIPULATION PART FOR SURGICAL INSTRUMENT AND SURGICAL INSTRUMENT FOR ELECTROCAUTERY INCLUDING THE MANIPULATION PART

(71) Applicant: LIVSMED INC., Seongnam-si (KR)

(72) Inventors: Chung Hyun LIM, Seoul (KR); Hee Jin KIM, Seoul (KR); Jwa Nyun KIM, Yongin-si (KR); Jae Yeong LEE, Seongnam-si (KR)

(21) Appl. No.: 19/189,051

(22) Filed: Apr. 24, 2025

(30) Foreign Application Priority Data
Apr. 26, 2024 (KR) 10-2024-0056031

(51) Int. Cl. A61B 18/14; A61B 18/00

(52) U.S. Cl. CPC A61B 18/1445; A61B 18/1482; A61B 2018/00202; A61B 2018/00595; A61B 2018/1455

(57) ABSTRACT

Provided is a manipulation part for a surgical instrument including an end tool, the manipulation part including a handle that is grippable, and an actuation manipulation part formed on one side of the handle and configured to control an actuation motion of the end tool, wherein the actuation manipulation part includes an actuation lever that is rotatable around an actuation rotation shaft, and an elastic member that is arranged between the handle and the actuation lever to be elastically deformable, and provides a restoring force to the actuation lever, and the elastic member includes a slide portion that is slidable while in contact with one of the handle and the actuation lever.

### CROSS-REFERENCE TO RELATED APPLICATION

This application is based on and claims priority under 35 U.S.C. § 119 to Korean Patent Application No. 10-2024-0056031, filed on Apr. 26, 2024, in the Korean Intellectual Property Office, the disclosure of which is incorporated by reference herein in its entirety. BACKGROUND

### 1. Field

The present disclosure relates to a surgical instrument for electrocautery, and more particularly, to a surgical instrument for electrocautery with improved insulation performance, which is mountable on a robotic arm or manually operable for use in laparoscopic surgery or various other surgeries.

### 2. Description of the Related Art

Surgical operations often require cutting and joining of body tissues, including organs, muscle tissues, connective tissues, and blood vessels. Over the centuries, sharp blades and sutures have been used for cutting and joining. However, during surgery, cutting of body tissues, especially highly vascularized tissues, results in bleeding. Therefore, surgeons have needed surgical instruments and methods to slow down or reduce bleeding during surgery.

Recently, electrosurgical instruments that use electrical energy have become available for certain surgical tasks. For example, electrosurgical instruments have been developed that include one or more electrodes configured to be supplied with electrical energy in surgical instruments such as graspers, scissors, forceps, blades, needles, or hooks. The electrical energy supplied through the electrodes may be used to coagulate, bond, or cut a patient's body tissue. In particular, when electrical energy is used, cutting and hemostasis may be performed simultaneously.

Electrosurgical instruments are typically classified into two types: monopolar and bipolar. In monopolar electrosurgical instruments, electrical energy of a specific polarity is supplied to one or more electrodes of the instrument. Electrical energy of the opposite polarity is electrically connected to a patient. In bipolar electrosurgical instruments, one or more electrodes are electrically connected to a first polarity electrical energy source, and one or more electrodes are electrically connected to a second polarity electrical energy source, which is opposite to the first polarity.

The above-mentioned background art is technical information possessed by the inventor for the derivation of the present disclosure or acquired during the derivation of the present disclosure, and cannot necessarily be said to be a known technique disclosed to the general public prior to the filing of the present disclosure.

### SUMMARY

The present invention provides a surgical instrument that is mountable on a robotic arm or manually operable for use in laparoscopic surgery or various other surgeries, the surgical instrument including an elastic member that provides a restoring force to allow an actuation lever returns to an initial position after performing grip and sealing motions.

Additional aspects will be set forth in part in the description which follows and, in part, will be apparent from the description, or may be learned by practice of the presented embodiments of the disclosure.

An embodiment of the present disclosure provides a manipulation part for a surgical instrument including an end tool, the manipulation part including a handle that is grippable, and an actuation manipulation part formed on one side of the handle and configured to control an actuation motion of the end tool, wherein the actuation manipulation part includes an actuation lever that is rotatable around an actuation rotation shaft, and an elastic member that is arranged between the handle and the actuation lever to be elastically deformable, and provides a restoring force to the actuation lever, and the elastic member includes a slide portion that is slidable while in contact with one of the handle and the actuation lever.

In an embodiment of the present disclosure, in response to the actuation lever rotating in one direction, the elastic member may be elastically deformed to provide the restoring force to cause the actuation lever to rotate in another direction.

In an embodiment of the present disclosure, one of the handle and the actuation lever, which comes into contact with the elastic member, may include a slide guide that guides sliding of the slide portion of the elastic member.

In an embodiment of the present disclosure, the slide guide may be formed to be recessed from one side of one of the handle and the actuation lever, which comes into contact with the elastic member, and a portion of the elastic member may be inserted into the slide guide to be movable along the slide guide.

In an embodiment of the present disclosure, in the slide guide, at least a portion of a contact surface that comes into contact with the slide portion may be formed to have a certain curvature.

In an embodiment of the present disclosure, the contact surface may include a first area having a constant inclination, and a second area extending from the first area and having a curvature.

In an embodiment of the present disclosure, the slide portion of the elastic member may slide along the first area and the second area, to reduce an amount of elastic deformation according to rotation of the actuation lever.

In an embodiment of the present disclosure, the elastic member may be a compression spring having one end slidably supported by one of the handle and the actuation lever, and another end fixed to the other one of the handle and the actuation lever.

In an embodiment of the present disclosure, the elastic member may be a torsion spring including a first spring arm extending from a coil portion to be connected to the handle, and a second spring arm extending from the coil portion to be connected to the actuation lever.

In an embodiment of the present disclosure, the coil portion may be arranged to be spaced apart from the actuation rotation shaft.

An embodiment of the present disclosure provides a surgical instrument for electrocautery including an end tool that is rotatable in at least one direction, a manipulation part including a handle that is grippable, and an actuation manipulation part that is formed on one side of the handle and configured to control an actuation motion of the end tool, and a power transmission part that includes an actuation wire connecting the end tool to the actuation manipulation part, and is configured to transmit power from the actuation manipulation part to the end tool, wherein the actuation manipulation part includes an actuation lever that is rotatable around an actuation rotation shaft, and an elastic member that is arranged between the handle and the actuation lever to be elastically deformable, and provides a restoring force to the actuation lever to offset tension of the actuation wire.

In an embodiment of the present disclosure, in response to the actuation lever rotating in one direction, the elastic member may be elastically deformed to provide the restoring force to cause the actuation lever to rotate in another direction.

In an embodiment of the present disclosure, the elastic member may include a slide portion that is slidable while in contact with one of the handle and the actuation lever.

In an embodiment of the present disclosure, one of the handle and the actuation lever, which comes into contact with the elastic member, may include a slide guide that comes into contact with the slide portion of the elastic member to guide sliding of the slide portion.

In an embodiment of the present disclosure, the slide guide may be formed to be recessed from one side of one of the handle and the actuation lever, which comes into contact with the elastic member, and a portion of the elastic member may be inserted into the slide guide to be movable along the slide guide.

In an embodiment of the present disclosure, in the slide guide, at least a portion of a contact surface that comes into contact with the slide portion may be formed to have a certain curvature.

In an embodiment of the present disclosure, the contact surface may include a first area having a constant inclination, and a second area extending from the first area and having a curvature.

In an embodiment of the present disclosure, the slide portion of the elastic member may slide along the first area and the second area, to reduce an amount of elastic deformation according to rotation of the actuation lever.

In an embodiment of the present disclosure, the elastic member may be a compression spring having one end slidably supported by one of the handle and the actuation lever, and another end fixed to the other one of the handle and the actuation lever.

In an embodiment of the present disclosure, the elastic member may be a torsion spring including a first spring arm extending from a coil portion to be connected to the handle, and a second spring arm extending from the coil portion to be connected to the actuation lever.

In an embodiment of the present disclosure, the coil portion may be arranged to be spaced apart from the actuation rotation shaft.

Other aspects, features, advantages other than those described above will become apparent from the following drawings, claims, and detailed description of the present disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a surgical instrument for electrocautery according to an embodiment of the present disclosure;

FIG. 2 is a perspective view illustrating the surgical instrument for electrocautery of FIG. 1;

FIGS. 3 and 4 are perspective views illustrating a manipulation part of the surgical instrument for electrocautery of FIG. 1;

FIG. 5 is a diagram schematically illustrating only a configuration of pulleys and wires constituting joints of the surgical instrument for electrocautery of FIG. 1;

FIG. 6 is a perspective view illustrating a yaw motion of the surgical instrument for electrocautery of FIG. 2;

FIGS. 7 and 8 are diagrams illustrating in detail a configuration of pulleys and wires associated with an actuation motion and a yaw motion of the surgical instrument for electrocautery illustrated in FIG. 1, with respect to a first jaw and a second jaw, respectively;

FIG. 9 is a perspective view illustrating a pitch motion of the surgical instrument for electrocautery of FIG. 1;

FIG. 10 and FIG. 11 are diagrams illustrating in detail a configuration of pulleys and wires associated with a pitch motion of the surgical instrument for electrocautery illustrated in FIG. 1, with respect to the first jaw and the second jaw, respectively;

FIGS. 12 and 13 are perspective views illustrating an operation of an actuation lever of the surgical instrument for electrocautery illustrated in FIG. 1;

FIGS. 14A and 14B and FIGS. 15A and 15B are diagrams illustrating movements of wires during an operation of the actuation lever of the surgical instrument for electrocautery illustrated in FIG. 1;

FIG. 16 is a diagram illustrating an elastic member of a surgical instrument for electrocautery according to an embodiment of the present disclosure;

FIG. 17 is a diagram illustrating a state in which an elastic member is elastically deformed during an operation of an actuation lever of the surgical instrument for electrocautery of FIG. 16;

FIGS. 18 and 19 are diagrams illustrating a modified example of the elastic member of FIG. 16 and FIG. 17;

FIGS. 20 and 21 are diagrams illustrating another modified example of the elastic member of FIG. 16 and FIG. 17;

FIGS. 22 and 23 are diagrams illustrating another embodiment of the elastic member of FIGS. 16 and 17;

FIGS. 24 and 25 are diagrams illustrating a modified example of the elastic member of FIGS. 22 and 23; and

FIGS. 26 and 27 are diagrams illustrating another embodiment of the elastic member of FIGS. 16 and 17.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments will be described in detail with reference to the accompanying drawings, and the same or corresponding components will be denoted by the same reference numerals when described with reference to the accompanying drawings, and thus their descriptions that are already provided will be omitted.

As the embodiments may be variously modified, particular embodiments will be illustrated in the drawings and described in detail in the detailed description. The effects and features of the present embodiments and methods of achieving them will become clear with reference to detailed descriptions provided below with the drawings. However, the embodiments are not limited to the descriptions below, and may be implemented in various forms.

In describing the present disclosure, detailed explanations of the related art are omitted when it is deemed that they may unnecessarily obscure the gist of the present disclosure.

In the following embodiments, the singular expression also includes the plural meaning as long as it is not inconsistent with the context. In the following embodiments, terms such as “first” or “second” may be used to describe various elements, but the elements should not be limited by the terms. These terms are used only to distinguish one element from another.

In the following embodiments, the terms “comprises,” “includes,” “has”, and the like used herein specify the presence of stated features or components, but do not preclude the presence or addition of one or more other features or components.

In the following embodiments, when a unit, region, or component is referred to as being “on” another unit, region, or component, it may be directly or indirectly on the other unit, region, or component, that is, one or more intervening units, regions, or components may be present therebetween.

In the following embodiments, when a component is referred to as being “connected to” or “coupled to” another component, the component may be directly connected to or in direct contact with the other component or intervening components may be present therebetween, unless clearly defined otherwise in the context.

For convenience of description, the magnitude of components in the drawings may be exaggerated or reduced. For example, each component in the drawings is illustrated to have an arbitrary size and thickness for ease of description, and thus the embodiments are not limited to the drawings.

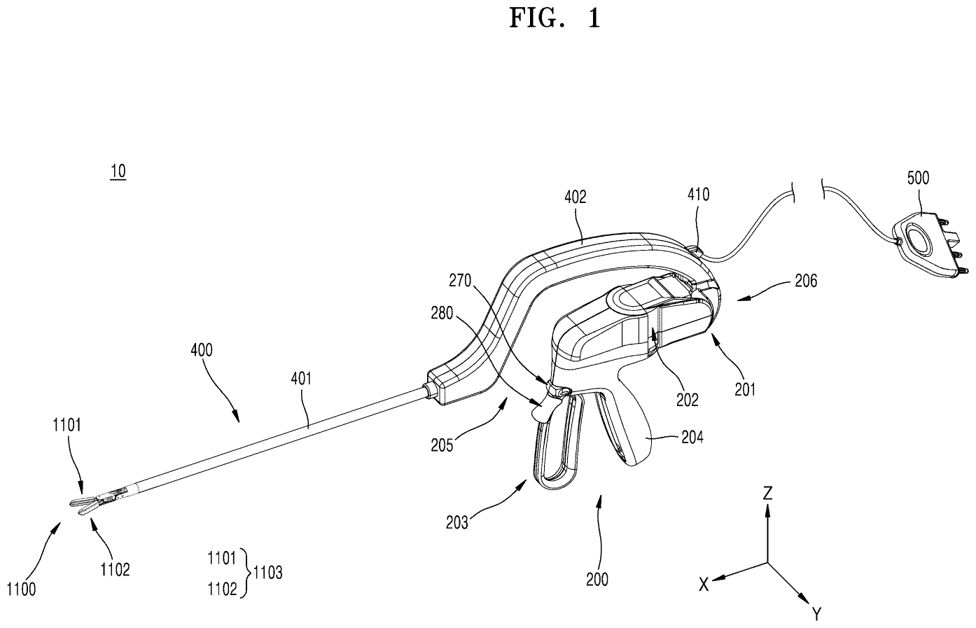

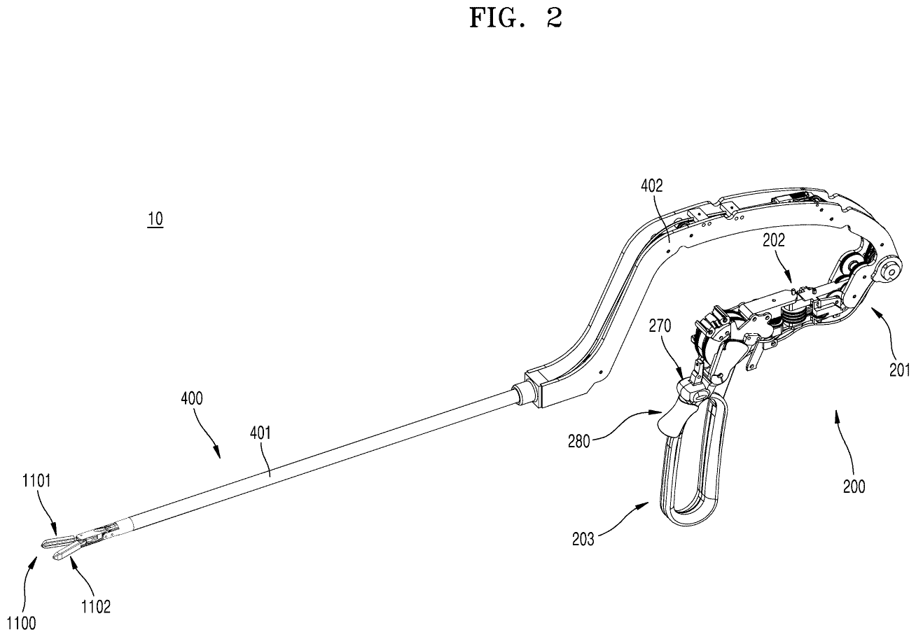

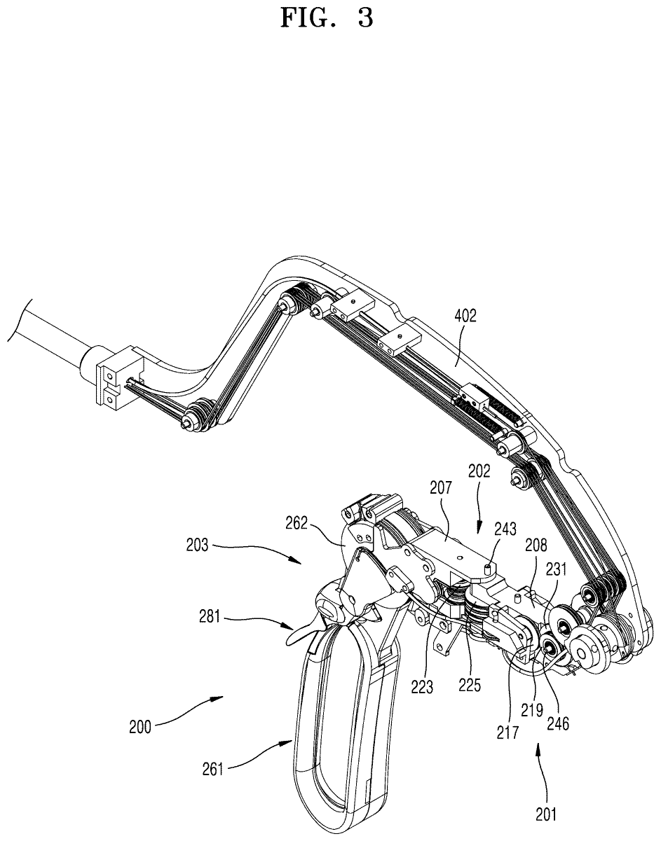

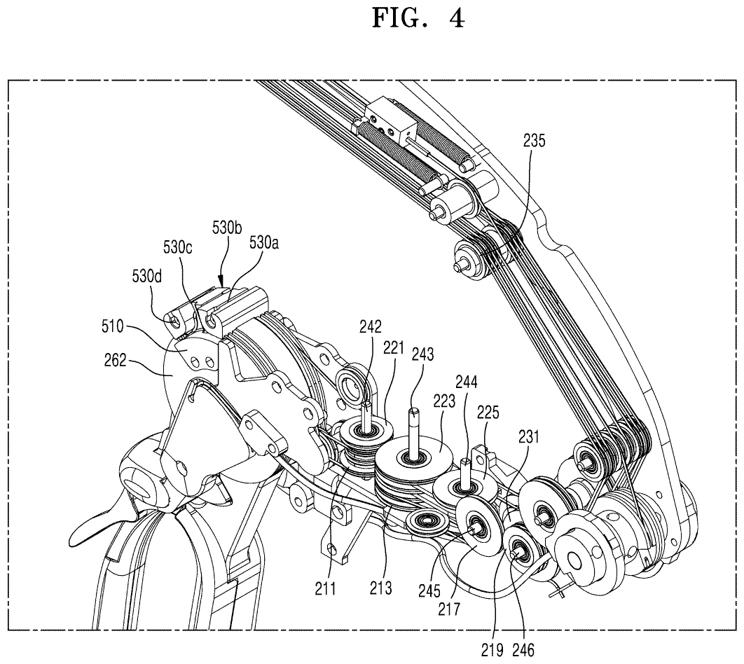

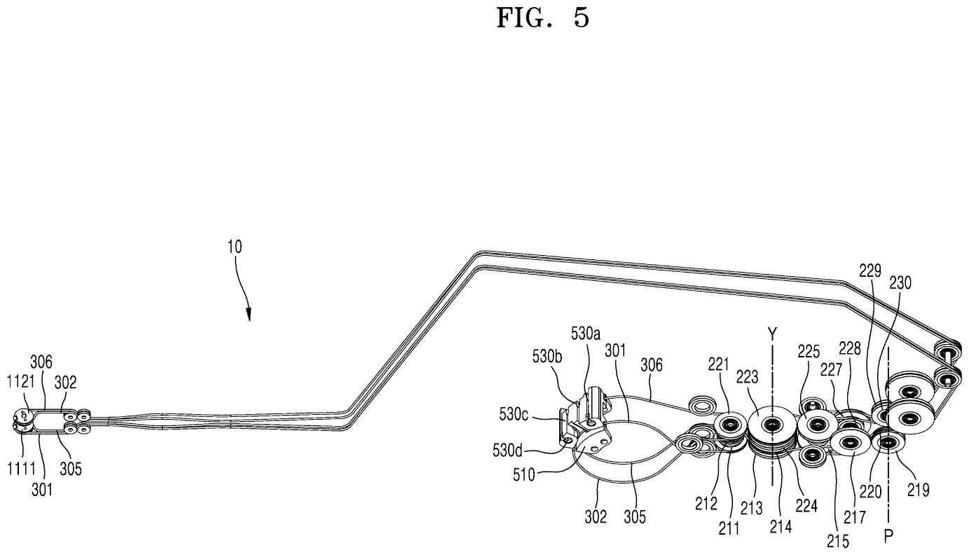

FIG. 1 is a perspective view illustrating a surgical instrument for electrocautery according to an embodiment of the present disclosure. FIG. 2 is a perspective view illustrating the surgical instrument for electrocautery of FIG. 1, and FIGS. 3 and 4 are perspective views illustrating a manipulation part of the surgical instrument for electrocautery of FIG. 1. In addition, FIG. 5 is a diagram schematically illustrating only a configuration of pulleys and wires constituting joints of the surgical instrument for electrocautery of FIG. 1.

Referring to FIGS. 1 to 5, a surgical instrument 10 for electrocautery according to an embodiment of the present disclosure includes an end tool 1100, a manipulation part 200, a power transmission part 300, and a connection part 400.

The connection part 400 may be formed in the shape of a hollow shaft, to accommodate therein one or more wires and electric wires. As the manipulation part 200 is coupled to one end of the connection part 400, and the end tool 1100 is coupled to the other end, the connection part 400 may serve to connect the manipulation part 200 to the end tool 1100. Here, the connection part 400 of the surgical instrument 10 for electrocautery according to an embodiment of the present disclosure includes a straight portion 401 and a bent portion 402. The straight portion 401 may be formed on the side coupled to the end tool 1100, and the bent portion 402 may be formed on the side coupled to the manipulation part 200. As such, as the end of the connection part 400 on the side of the manipulation part 200 is formed to be bent, a pitch manipulation part 201, a yaw manipulation part 202, and an actuation manipulation part 203 are formed along or adjacent to the extension line of the end tool 1100. In other words, it may also be described that the pitch manipulation part 201 and the yaw manipulation part 202 are at least partially accommodated in a concave portion formed by the bent portion 402. Through the above-described shape of the bent portion 402, the shapes and motions of the manipulation part 200 and the end tool 1100 may match each other more intuitively.

In addition, a plane on which the bent portion 402 is formed may be a pitch plane, that is, a plane substantially the same as an XZ plane of FIG. 1. As such, as the bent portion 402 is formed on a plane substantially the same as the XZ plane, interference with the manipulation part may be reduced. Obviously, for intuitive motions of the end tool and the manipulation part, any form other than the XZ plane may be possible.

In addition, a connector 410 may be formed in the bent portion 402. The connector 410 may be connected to an external power source (not shown), and the connector 410 may also be connected to jaws 1103 via electric wires, to transmit, to the jaws 1103, electric energy supplied from the external power source (not shown). Here, the connector 410 may be of a bipolar type with two electrodes formed therein, or may be of a monopolar type with one electrode formed therein.

The manipulation part 200 may be formed at one end of the connection part 400 and may include an interface that may be directly manipulated by a doctor, for example, an interface in the shape of a pincer, a stick, a lever, etc. When the doctor manipulates the interface, the end tool 1100, which is connected to the interface to be inserted into a patient's body, operates in a certain manner to perform surgery. Here, although FIG. 1 illustrates that the manipulation part 200 is formed in the shape of a handle that may be rotated while fingers are inserted therein, the present disclosure is not limited thereto, and various types of manipulation parts that may be connected to the end tool 1100 to manipulate the end tool 1100 may be possible.

The end tool 1100 may be formed at the other end of the connection part 400 and may be inserted into a surgical site to perform a motion necessary for surgery. As an example of the end tool 1100, as illustrated in FIG. 2, the end tool 1100 may include a pair of jaws for performing a grip motion, that is, a first jaw 1101 and a second jaw 1102. Here, a component encompassing each of the first jaw 1101 and the second jaw 1102 or both the first jaw 1101 and the second jaw 1102 may be referred to as a jaw 1103.

However, the concept of the present disclosure is not limited thereto, and various other surgical instruments may be used as the end tool 1100. For example, a one-armed cautery may be used as the end tool. As the end tool 1100 is connected to the manipulation part 200 by the power transmission part 300, the end tool 1100 may receive a driving force of the manipulation part 200 through the power transmission part 300 to perform motions required for surgery, such as a grip motion, a cutting motion, or a suturing motion.

Here, the end tool 1100 of the surgical instrument 10 for electrocautery according to an embodiment of the present disclosure may be formed to be rotatable in at least one direction, and for example, the end tool 1100 may be formed to perform a pitch motion around the Y-axis of FIG. 1 and simultaneously perform a yaw motion and an actuation motion around the Z-axis of FIG. 1.

In addition, the end tool 1100 may include a pulley 1111 and the like associated with a rotational motion of the first jaw 1101. In addition, the end tool 1100 may include a pulley 1121 and the like associated with a rotational motion of the second jaw 1102.

The power transmission part 300 may serve to connect the manipulation part 200 to the end tool 1100 to transmit a driving force of the manipulation part 200 to the end tool 1100, and may include a plurality of wires, pulleys, links, joints, gears, and the like.

Referring to FIG. 5, the power transmission part 300 of the surgical instrument 10 for electrocautery according to an embodiment of the present disclosure may include a wire 301, a wire 302, a wire 305, and a wire 306.

Here, the wire 301 and the wire 305 may be paired to serve as first jaw wires. The wire 302 and the wire 306 may be paired to serve as second jaw wires. Here, components including the wire 301 and the wire 305, which are first jaw wires, and the wire 302 and the wire 306, which are second jaw wires, may be referred to as jaw wires. In addition, a pair of wires (not shown) may serve as pitch wires.

In addition, the power transmission part 300 of the surgical instrument 10 for electrocautery according to an embodiment of the present disclosure may include fastening members (not shown) coupled to ends of wires so as to couple the wires to pulleys, respectively. Here, each of the fastening members may have various shapes as necessary, such as a ball shape or a tube shape.

The coupling relationships between the wires, the fastening members, and the pulleys will be described in detail below.

First, the wire 301 and the wire 305, which are first jaw wires, may constitute a single wire. When a fastening member (not shown), which is a first jaw wire-end tool fastening member, is fit into a middle point of the single first jaw wire, and the fastening member (not shown) is fixed through crimping, both strands of the first jaw wire around the fastening member (not shown) may be referred to as the wire 301 and the wire 305, respectively.

Alternatively, the wire 301 and the wire 305, which are first jaw wires, may be formed as separate wires and then connected to each other by the fastening member (not shown).

Then, by coupling the fastening member (not shown) to the pulley 1111, the wire 301 and the wire 305 may be fixedly coupled to the pulley 1111. Accordingly, the pulley 1111 may rotate as the wire 301 and the wire 305 are pulled and released.

In addition, a first jaw wire-manipulation part fastening member may be coupled to ends of the wire 301 and the wire 305 that are opposite to the ends to which the first jaw wire-end tool fastening member is coupled.

In addition, by connecting the first jaw wire-manipulation part fastening member to a pulley 211 in this manner, the wire 301 and the wire 305 may be fixedly connected to the pulley 211. Accordingly, when the pulley 211 is rotated by a motor or a human force, the pulley 1111 of the end tool 1100 may rotate as the wire 301 and the wire 305 are pulled and released.

In the same manner, the wire 302 and the wire 306, which are second jaw wires, are coupled to a second jaw wire-end tool fastening member and a second jaw wire-manipulation part fastening member, respectively. In addition, the second jaw wire-end tool fastening member is coupled to the pulley 1121, and the second jaw wire-manipulation part fastening member is coupled to a pulley 220. Accordingly, when the pulley 220 is rotated by a motor or a human force, the pulley 1121 of the end tool 1100 may rotate as the wire 302 and the wire 306 are pulled and released.

In the same manner, pitch wires (not shown) are coupled to a pitch wire-end tool fastening member and a pitch wire-manipulation part fastening member, respectively.

### Manipulation Part

Hereinafter, the manipulation part 200 of the surgical instrument 10 for electrocautery of FIG. 1 will be described in more detail.

Referring to FIGS. 1 to 5, the manipulation part 200 of the surgical instrument 10 for electrocautery according to an embodiment of the present disclosure includes a handle 204 that may be held by a user, the actuation manipulation part 203 configured to control an actuation motion of the end tool 1100, the yaw manipulation part 202 configured to control a yaw motion of the end tool 1100, and the pitch manipulation part 201 configured to control a pitch motion of the end tool 1100. Here, it may be understood that FIGS. 3 and 4 illustrate only components associated with pitch/yaw/actuation motions of the surgical instrument 10 for electrocautery.

In addition, the manipulation part 200 of the surgical instrument 10 for electrocautery further includes a blade manipulation part 280 configured to control a motion of a blade of the end tool 1100 to perform cutting, and a sealing manipulation part 270 configured to control supply of electric energy to an electrode (not shown) of the end tool 1100 to perform cauterization.

The manipulation part 200 may include the pulley 211, a pulley 212, a pulley 213, a pulley 214, a pulley 215, a pulley 217, a pulley 218, a pulley 219, and the pulley 220, which are associated with a rotational motion of the first jaw 1101. In addition, the manipulation part 200 may include a pulley 221, a pulley 222, a pulley 223, a pulley 224, a pulley 225, a pulley 227, a pulley 228, a pulley 229, and a pulley 230, which are associated with a rotational motion of the second jaw 1102. In addition, the manipulation part 200 may include a pulley 262 associated with rotational motions of the first jaw and the second jaw. In addition, the manipulation part 200 may include a pulley 231 associated with a pitch motion. In addition, the manipulation part 200 may include relay pulleys 235 arranged in intermediate points of the bent portion 402 of the connection part 400.

Here, although the drawings illustrate that the pulleys facing each other are arranged in parallel to each other, the present disclosure is not limited thereto, and the pulleys may be formed in various positions and sizes suitable for the configuration of the manipulation part.

In addition, the manipulation part 200 according to an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a manipulation part actuation rotation shaft, and the rotation shaft 242 may function as a manipulation part first yaw sub-rotation shaft. Here, the rotation shaft 243 may function as a manipulation part yaw main rotation shaft, and the rotation shaft 244 may function as a manipulation part second yaw sub-rotation shaft. In addition, the rotation shaft 245 may function as a manipulation part pitch sub-rotation shaft, and the rotation shaft 246 may function as a manipulation part pitch main rotation shaft.

The rotation shaft 241, the rotation shaft 242, the rotation shaft 243, the rotation shaft 244, the rotation shaft 245, and the rotation shaft 246 may be arranged sequentially in the direction from a distal end 205 of the manipulation part 200 toward a proximal end 206.

The pulley 262 may function as an actuation pulley for the first jaw and the second jaw, and may be referred to as a manipulation part actuation pulley.

The pulley 211 and the pulley 212 may function as manipulation part first jaw first yaw sub-pulleys, the pulley 221 and the pulley 222 may function as manipulation part second jaw first yaw sub-pulleys, and these components may be collectively referred to as manipulation part first yaw sub-pulleys.

The pulley 213 and the pulley 214 may function as manipulation part first jaw yaw main pulleys, the pulley 223 and the pulley 224 may function as manipulation part second jaw yaw main pulleys, and these components may be collectively referred to as manipulation part yaw main pulleys.

The pulley 215 may function as a manipulation part first jaw second yaw sub-pulley, the pulley 225 may function as a manipulation part second jaw second yaw sub-pulley, and these components may be collectively referred to as manipulation part second yaw sub-pulleys.

The pulley 217 and the pulley 218 may function as manipulation part first jaw pitch sub-pulleys, the pulley 227 and the pulley 228 may function as manipulation part second jaw pitch sub-pulleys, and these components may be collectively referred to as manipulation part pitch sub-pulleys.

The pulley 219 and the pulley 220 may function as manipulation part first jaw pitch main pulleys, the pulley 229 and the pulley 230 may function as manipulation part second jaw pitch main pulleys, and these components may be collectively referred to as manipulation part pitch main pulleys.

The pulley 231 may function as a manipulation part pitch wire main pulley, and may include a pulley (not shown) that functions as a manipulation part pitch wire sub-pulley.

The above components are classified from the perspective of the manipulation part for each motion (pitch/yaw/actuation) as follows.

The pitch manipulation part 201 that controls a pitch motion of the end tool 1100 may include the pulley 217, the pulley 218, the pulley 219, the pulley 220, the pulley 227, the pulley 228, the pulley 229, the pulley 230, and the pulley 231. In addition, the pitch manipulation part 201 may include the rotation shaft 245 and the rotation shaft 246. In addition, the pitch manipulation part 201 may further include a pitch frame 208.

The yaw manipulation part 202 that controls a yaw motion of the end tool 1100 may include the pulley 211, the pulley 212, the pulley 213, the pulley 214, the pulley 215, the pulley 221, the pulley 222, the pulley 223, the pulley 224, and the pulley 225. In addition, the yaw manipulation part 202 may include the rotation shaft 242, the rotation shaft 243, and the rotation shaft 244. In addition, the yaw manipulation part 202 may further include a yaw frame 207.

The actuation manipulation part 203 that controls an actuation motion of the end tool 1100 may include the pulley 262 and the rotation shaft 241.

Hereinafter, each component of the manipulation part 200 will be described in more detail.

The handle 204 may be formed to be gripped by a user with his/her hand, and in particular, may be formed such that the user may grasp the handle 204 by wrapping his/her palm around the handle 204. In addition, the actuation manipulation part 203 and the yaw manipulation part 202 are formed on the handle 204, and the pitch manipulation part 201 is formed on one side of the yaw manipulation part 202. In addition, the other end of the pitch manipulation part 201 is connected to the bent portion 402 of the connection part 400.

The actuation manipulation part 203 includes the blade manipulation part 260. The blade manipulation part 260 may include an actuation lever 261 and an actuation pulley 262.

Here, the actuation lever 261 may be formed in the shape of a hand ring, and may function as a second handle.

Here, the rotation shaft 241, which is an actuation rotation shaft, may be formed to form a certain angle with the XZ plane on which the connection part 400 is formed.

For example, the rotation shaft 241 may be formed in a direction parallel to the Y-axis, and in this state, when the pitch manipulation part 201 or the yaw manipulation part 202 rotates, the coordinate system of the actuation manipulation part 203 may change relatively. Obviously, the concept of the present disclosure is not limited thereto, and the rotation shaft 241 may be formed in various directions to suit the hand structure of the user gripping the actuation manipulation part 203, through ergonomic design.

In addition, the actuation pulley 262 may be coupled to the actuation lever 261 or may be formed as a single member. Thus, as the actuation lever 261 rotates, the actuation pulley 262 may rotate together.

Here, the actuation pulley 262 may be composed of one pulley or may be composed of two pulleys fixedly coupled to each other.

The yaw manipulation part 202 may include the rotation shaft 242, the rotation shaft 243, the pulley 213 and the pulley 214, which are manipulation part first jaw yaw main pulleys, the pulley 223 and the pulley 224, which are manipulation part second jaw yaw main pulleys, and the yaw frame 207. In addition, the yaw manipulation part 202 may further include the pulley 211 and the pulley 212, which are manipulation part first jaw first yaw sub-pulleys formed on one side of the pulley 213 and the pulley 214, and the pulley 221 and the pulley 222, which are manipulation part second jaw first yaw sub-pulleys formed on one side of the pulley 223 and the pulley 224. In addition, the yaw manipulation part 202 may further include the pulley 215, which is a manipulation part first jaw second yaw sub-pulley formed on another side of the pulley 213 and the pulley 214, and the pulley 225, which is a manipulation part second jaw second yaw sub-pulley formed on another side of the pulley 223 and the pulley 224. Here, the pulley 215 and the pulley 225 may be coupled to the pitch frame 208 to be described below.

Here, the drawings illustrate that the yaw manipulation part 202 includes the pulley 213, the pulley 214, the pulley 223, and the pulley 224, and that the pulley 213 and the pulley 214 are formed to face each other, the pulley 223 and the pulley 224 are formed to face each other, and thus the yaw manipulation part 202 have two pulleys that independently rotatable, but the concept of the present disclosure is not limited thereto. That is, one or more pulleys having the same or different diameters may be provided according to the configuration of the yaw manipulation part 202.

In detail, on the handle 204, the rotation shaft 242, which is a manipulation part first yaw sub-rotation shaft, is formed on one side of the actuation manipulation part 203, and the rotation shaft 243, which is a manipulation part yaw main rotation shaft, is formed on one side of the rotation shaft 242. Here, the handle 204 is formed to be rotatable around the rotation shaft 243.

Here, the rotation shaft 243 may be formed to have a certain angle with the XY plane on which the connection part 400 is formed. For example, the rotation shaft 243 may be formed in a direction parallel to the Z-axis, and when the pitch manipulation part 201 rotates in this state, the coordinate system of the rotation shaft 243 may relatively change as described above. Obviously, the concept of the present disclosure is not limited thereto, and the rotation shaft 243 may be formed in various directions to suit the hand structure of the user gripping the manipulation part 200, through ergonomic design.

In addition, the pulley 213, the pulley 214, the pulley 223, and the pulley 224 are coupled to the rotation shaft 243 to be rotatable around the rotation shaft 243. In addition, the wire 301 or the wire 305, which is a first jaw wire, may be wound around the pulley 213 and the pulley 214, and the wire 302 or the wire 306, which is a second jaw wire, may be wound around the pulley 223 and the pulley 224. Here, the pulley 213 and the pulley 214 may be formed to face each other and the pulley 223 and the pulley 224 may be formed to face each other, so as to form two independently rotatable pulleys. Accordingly, the wires being wound inward and the wires being wound outward may be respectively wound around separate pulleys, and thus may operate without interfering with each other.

Because the yaw frame 207 rigidly connects the handle 204, the rotation shaft 242, and the rotation shaft 243 to each other, and the actuation manipulation part 203 in which the rotation shaft 241 and the actuation pulley 262 are combined with each other is rigidly connected to the yaw frame 207 directly or through an intermediary member, the handle 204, the yaw manipulation part 202, and the actuation manipulation part 203 may integrally yaw-rotate around the rotation shaft 243.

The pitch manipulation part 201 may include the rotation shaft 246, the pulley 219 and a pulley 220, which are manipulation part first jaw pitch main pulleys, the pulley 229 and the pulley 230, which are manipulation part second jaw pitch main pulleys, and the pitch frame 208. In addition, the pitch manipulation part 201 may further include the rotation shaft 245, the pulley 217 and the pulley 218, which are manipulation part first jaw pitch sub-pulleys formed on one side of the pulley 219 and the pulley 220, and the pulley 227 and the pulley 228, which are manipulation part second jaw pitch sub-pulleys formed on one side of the pulley 229 and the pulley 230. The pitch manipulation part 201 may be connected to the bent portion 402 of the connection part 400 through the rotation shaft 246.

In detail, the pitch frame 208 may serve as a base frame of the pitch manipulation part 201, and the rotation shaft 243 is rotatably connected to one end of the pitch frame 208. That is, the yaw frame 207 is formed to be rotatable around the rotation shaft 243 with respect to the pitch frame 208.

As described above, because the yaw frame 207 connects the handle 204, the rotation shaft 243, the rotation shaft 241, and the rotation shaft 242 to each other, and the yaw frame 207 is axially coupled to the pitch frame 208, when the pitch frame 208 pitch-rotates around the rotation shaft 246, the yaw frame 207, the handle 204, the rotation shaft 241, the rotation shaft 242, and the rotation shaft 243 connected to the pitch frame 208 pitch-rotate together. That is, when the pitch manipulation part 201 rotates around the rotation shaft 246, the actuation manipulation part 203 and the yaw manipulation part 202 rotate together with the pitch manipulation part 201. In other words, when the user rotates the handle 204 around the rotation shaft 246, the actuation manipulation part 203, the yaw manipulation part 202, and the pitch manipulation part 201 move together with the handle 204.

The pulley 219, the pulley 220, the pulley 229, and the pulley 230 are coupled to the rotation shaft 246 so as to be rotatable around the rotation shaft 246 of the pitch frame 208.

Here, the pulley 219 and the pulley 220 may be formed to face each other and to be rotatable independently. Accordingly, the wires being wound inward and the wires being wound outward may be respectively wound around separate pulleys, and thus may operate without interfering with each other. Likewise, the pulley 229 and the pulley 230 may also be formed to face each other and to be rotatable independently. Accordingly, the wires being wound inward and the wires being wound outward may be respectively wound around separate pulleys, and thus may operate without interfering with each other.

The connection relationships between the handle 204, the pitch manipulation part 201, the yaw manipulation part 202, and the actuation manipulation part 203 are as follows. The rotation shaft 241, the rotation shaft 242, the rotation shaft 243, the rotation shaft 244, the rotation shaft 245, and the rotation shaft 246 may be formed on the handle 204. Here, because the rotation shaft 242 and the rotation shaft 243 are formed directly on the handle 204, the handle 204 and the yaw manipulation part 202 may be directly connected to each other. On the other hand, because the pitch manipulation part 201 is formed to be connected to the yaw manipulation part 202 on one side of the yaw manipulation part 202, the pitch manipulation part 201 may not be directly connected to the handle 204, and the pitch manipulation part 201 and the handle 204 may be formed to be indirectly connected to each other through the yaw manipulation part 202. In addition, because the actuation manipulation part 203 is formed to be connected to the yaw manipulation part 202 on another side of the yaw manipulation part 202, the actuation manipulation part 203 may not be directly connected to the handle 204, and the actuation manipulation part 203 and the handle 204 may be formed to be indirectly connected to each other through the yaw manipulation part 202.

Continuing to refer to the drawings, in the surgical instrument 10 for electrocautery according to an embodiment of the present disclosure, the pitch manipulation part 201 and the end tool 1100 may be formed on the same axis or parallel axes (X-axis). That is, the rotation shaft 246 of the pitch manipulation part 201 is formed at one end of the bent portion 402 of the connection part 400, and the end tool 1100 is formed at the other end of the connection part 400.

In addition, one or more relay pulleys 235 may be arranged in intermediate points of the connection part 400, particularly around the bent portion 402, to change the paths of the wires or to guide the wires along their paths. As such, the wires are formed to be wound at least in part around the relay pulleys 235 to guide the wires along their paths, and thus may be arranged along the bent shape of the bent portion 402.

Here, the drawings illustrate that the connection part 400 includes the bent portion 402 and thus is formed to be bent with a certain curvature, but the concept of the present disclosure is not limited thereto, and the connection part 400 may be formed as a straight line, or may be formed to be bent at least once as necessary. Even in this case, it may be described that the pitch manipulation part 201 and the end tool 1100 are formed on substantially the same or parallel axes. In addition, although FIG. 2 illustrates that the pitch manipulation part 201 and the end tool 1100 are formed on an axis parallel to the X-axis, the concept of the present disclosure is not limited thereto, and the pitch manipulation part 201 and the end tool 1100 may be formed on different axes.

### Actuation Motion, Yaw Motion, and Pitch Motion

An actuation motion, a yaw motion, and a pitch motion in the present embodiment are as follows.

First, the actuation motion is as follows.

When the user rotates the actuation lever 261 while placing his/her finger in a hand ring formed on the actuation lever 261, the actuation pulley 262 coupled to the actuation lever 261 rotates around the rotation shaft 241.

At this time, the wire 301 and the wire 305 wound around the pulley 262 with one end fixedly connected to the pulley 262, and the wire 302 and the wire 306 wound around the same pulley 262 with one end fixedly connected to the pulley 262 move as the pulley 262 rotates. Here, the wire 301, the wire 302, the wire 305, and the wire 306 are connected to one actuation pulley 262, but the movement of each of the wires according to the rotation of the pulley varies depending on the direction in which each wire is wound around the pulley 262. This will be described in detail below.

In addition, such a rotational force is transmitted to the end tool 1100 through the power transmission part 300, such that two jaws 1103 of the end tool 1100 perform an actuation motion.

Here, as described above, the actuation motion refers to a motion in which the two jaws 1011 and 1102 are opened or closed while rotating in opposite directions. That is, when the actuation lever 261 of the actuation manipulation part 203 is rotated toward the handle 204, the first jaw 1101 rotates counterclockwise and the second jaw 1102 rotates clockwise, thereby closing the end tool 1100. On the contrary, when the actuation lever 261 of the actuation manipulation part 203 is rotated away from the handle 204, the first jaw 1101 rotates clockwise and the second jaw 1102 rotates counterclockwise, thereby opening the end tool 1100.

Next, the yaw motion is as follows.

When the user gripping the handle 204 rotates the handle 204 around the rotation shaft 243, the actuation manipulation part 203 and the yaw manipulation part 202 rotate around the rotation shaft 243. That is, when the actuation pulley 262 to which the wire 301 and the wire 305 are fixedly connected rotates around the rotation shaft 243, the wire 301 and the wire 305 wound around the pulley 213 and the pulley 214 move. Likewise, because the wire 302 and the wire 306 are fixedly connected to the actuation pulley 262, when the actuation pulley 262 rotates around the rotation shaft 243, the wire 302 and the wire 306 wound around the pulley 223 and the pulley 224 move. At this time, the wire 301 and the wire 305 connected to the first jaw 1101, and the wire 302 and the wire 306 connected to the second jaw 1102 are wound around the pulley 213 and the pulley 214, and the pulley 223 and the pulley 224, respectively, such that the first jaw 1101 and the second jaw 1102 rotate in the same direction during yaw rotation. In addition, such a rotational force is transmitted to the end tool 1100 through the power transmission part 300, such that the two jaws 1103 of the end tool 1100 perform a yaw motion to rotate in the same direction.

At this time, because the yaw frame 207 connects the handle 204, the rotation shaft 241, the rotation shaft 242, and the rotation shaft 243 to each other, the handle 204, the yaw manipulation part 202, and the actuation manipulation part 203 rotate together around the rotation shaft 243.

Next, the pitch motion is as follows.

When the user gripping the handle 204 rotates the handle 204 around the rotation shaft 246, the actuation manipulation part 203, the yaw manipulation part 202, and the pitch manipulation part 201 pitch-rotate around the rotation shaft 246. That is, when the actuation pulley 262 to which the wire 301 and the wire 305 are fixedly connected rotates around the rotation shaft 246, the wire 301 and the wire 305 wound around the pulley 219 and the pulley 220 move. Likewise, when the actuation pulley 262 to which the wire 302 and the wire 306 are fixedly connected rotates around the rotation shaft 246, the wire 302 and the wire 306 wound around the pulley 229 and the pulley 230 move. At this time, as described above with reference to FIG. 5, etc., the wire 301, the wire 305, the wire 302, and the wire 306, which are jaw wires, are wound respectively around the pulley 219, the pulley 220, the pulley 229, and the pulley 230, which are manipulation part pitch main pulleys, such that the wire 301 and the wire 305, which are first jaw wires, move in the same direction, and the wire 302 and the wire 306, which are second jaw wires, move in the same direction so as to allow the first jaw 1101 and the second jaw 1102 to pitch-rotate. In addition, such a rotational force is transmitted to the end tool 1100 through the power transmission part 300, such that two jaws 1103 of the end tool 1100 perform a pitch motion.

At this time, because the pitch frame 208 is connected to the yaw frame 207, and the yaw frame 207 connects the handle 204, the rotation shaft 241, the rotation shaft 242, and the rotation shaft 243 to each other, when the pitch frame 208 rotates around the rotation shaft 246, the yaw frame 207, the handle 204, the rotation shaft 241, the rotation shaft 242, and the rotation shaft 243 connected to the pitch frame 208 rotate together. That is, when the pitch manipulation part 201 rotates around the rotation shaft 246, the actuation manipulation part 203 and the yaw manipulation part 202 rotate together with the pitch manipulation part 201.

In summary, in the surgical instrument 10 for electrocautery according to an embodiment of the present disclosure, a pulley is formed at each joint point (an actuation joint, a yaw joint, and a pitch joint), a wire (a first jaw wire or a second jaw wire) is wound around the pulley, and rotational manipulation of the manipulation part (actuation rotation, yaw rotation, and pitch rotation) causes a movement of each wire, thereby inducing a desired motion of the end tool 1100. Furthermore, auxiliary pulleys may be formed on one side of each pulley, and these auxiliary pulleys may prevent the wires from being wound multiple times around one pulley.

FIG. 5 is a diagram schematically illustrating only a configuration of pulleys and wires constituting joints of the surgical instrument 10 for electrocautery illustrated in FIG. 1, according to an embodiment of the present disclosure. In FIG. 5, relay pulleys for changing paths of wires, which are not associated with motions of the joints, are omitted.

Referring to FIG. 5, the manipulation part 200 may include the pulley 211, the pulley 212, the pulley 213, the pulley 214, the pulley 215, the pulley 217, the pulley 218, the pulley 219, and the pulley 220, which are associated with a rotational motion of the first jaw 1101.

In addition, the manipulation part 200 may include the pulley 221, the pulley 222, the pulley 223, the pulley 224, the pulley 225, the pulley 227, the pulley 228, the pulley 229, and the pulley 230, which are associated with a rotational motion of the second jaw 1102. In addition, the manipulation part 200 may include the pulley 262 associated with rotational motions of the first jaw and the second jaw (The arrangement and configuration of each pulley in the manipulation part 200 is the same as the arrangement and configuration of each pulley in the end tool 1100 in principle, and thus, some of reference numerals thereof are omitted in the drawings).

The pulley 211, the pulley 212, the pulley 221, and the pulley 222 may be formed to be rotatable independently of each other around the same shaft, that is, the rotation shaft 242. Here, the pulley 211 and the pulley 212 may be formed as two pulleys that face each other and are rotatable independently of each other. Likewise, the pulley 221 and the pulley 222 may be formed as two pulleys that face each other and are rotatable independently of each other, and in this case, the two pulleys may be formed to have different diameters.

The pulley 213, the pulley 214, the pulley 223, and the pulley 224 may be formed to be rotatable independently of each other around the same axis, that is, the rotation shaft 243. Here, the pulley 213 and the pulley 214, and the pulley 223 and the pulley 224 may be respectively formed as two pulleys that face each other and are rotatable independently of each other.

The pulley 215 and the pulley 225 may be formed to be rotatable independently of each other around the same axis, that is, the rotation shaft 244.

The pulley 217, the pulley 218, and the pulley 227, and the pulley 228 may be formed to be rotatable independently of each other around the same axis, that is, the rotation shaft 245. Here, the pulley 217 and the pulley 218 may be formed to have different diameters. In addition, the pulley 227 and the pulley 228 may be formed to have different diameters.

The pulley 219, the pulley 220, the pulley 229, and the pulley 230 may be formed to be rotatable independently of each other around the same shaft, that is, the rotation shaft 246.

The wire 301 passes sequentially through the pulley 219, the pulley 217, the pulley 215, the pulley 213, and the pulley 211 of the manipulation part 200, is then wound around the pulley 262, and is then connected to the pulley 262 by a fastening member 530b. In addition, the wire 305 passes sequentially through the pulley 220, the pulley 218, the pulley 214, and the pulley 212 of the manipulation part 200, and is then connected to the pulley 262 by the fastening member 530d. Accordingly, when the pulley 262 rotates, the wire 301 and the wire 305 are wound around or unwound from the pulley 262, causing the first jaw 1101 to rotate.

The wire 306 passes sequentially through the pulley 229, the pulley 227, the pulley 225, the pulley 223, and the pulley 221 of the manipulation part 200, and is then wound around the pulley 262, and is then connected to the pulley 262 by the fastening member 530a. In addition, the wire 302 passes sequentially through the pulley 230, the pulley 228, the pulley 224, and the pulley 222 of the manipulation part 200, and is then connected to the pulley 262 by the fastening member 530c. Accordingly, when the pulley 262 rotates, the wire 302 and the wire 306 are wound around or unwound from the pulley 262, causing the second jaw 1102 to rotate.

### Conceptual Diagram of Pulleys and Wires

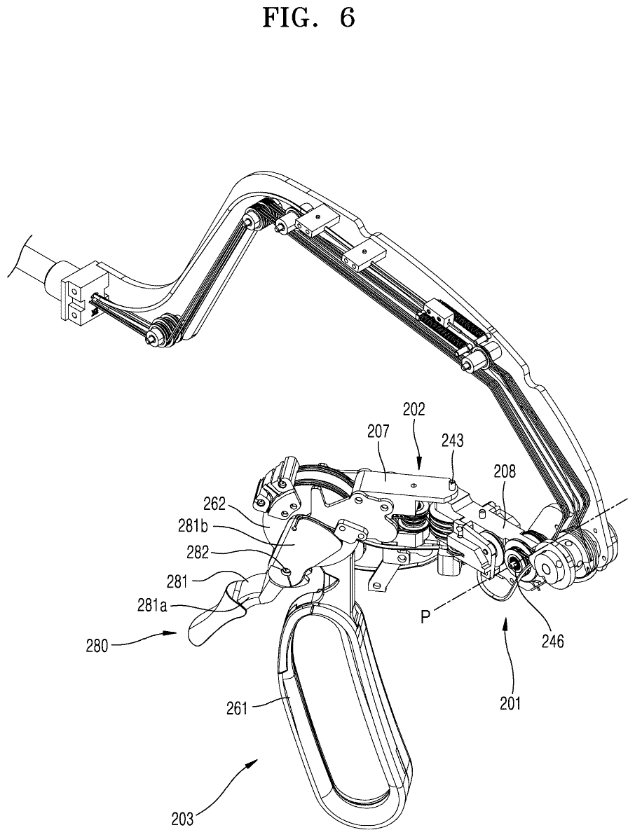

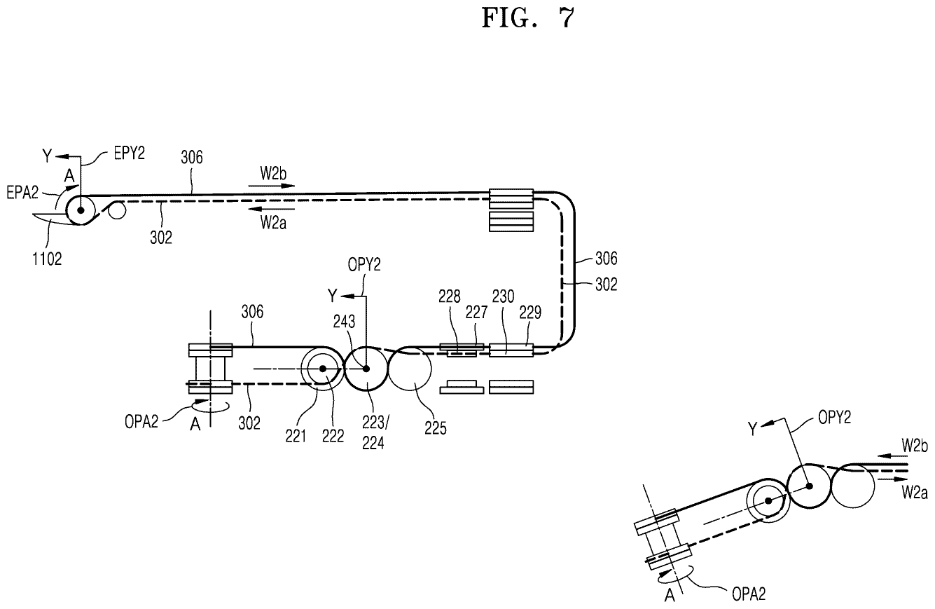

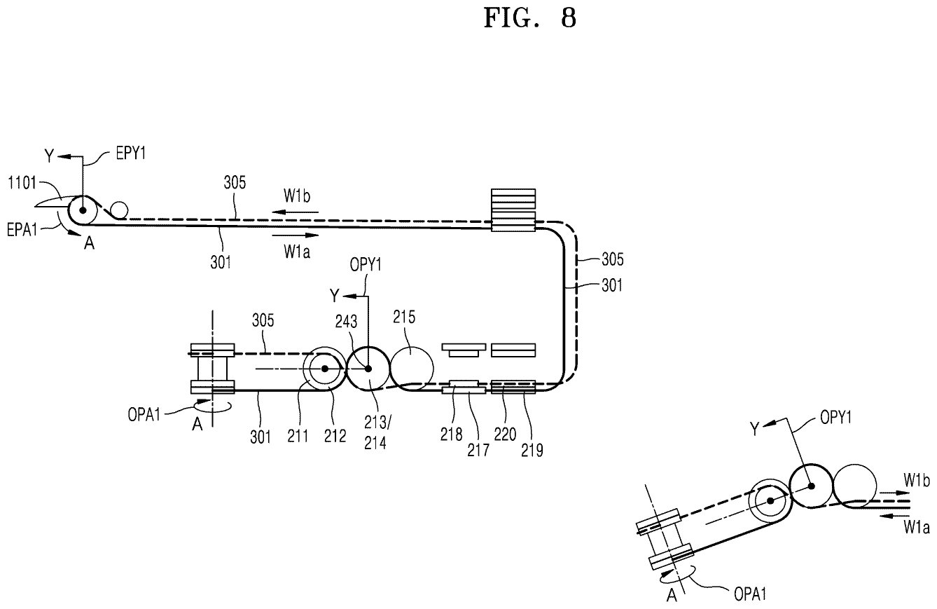

FIGS. 7 and 8 are diagrams illustrating in detail a configuration of pulleys and wires associated with an actuation motion and a yaw motion of the surgical instrument 10 for electrocautery illustrated in FIG. 1, with respect to the first jaw and the second jaw, respectively, according to an embodiment of the present disclosure. FIG. 7 is a diagram illustrating only pulleys and wires associated with the second jaw, and FIG. 8 is a diagram illustrating only pulleys and wires associated with the first jaw. In addition, FIG. 6 is a perspective view illustrating a yaw motion of the surgical instrument of FIG. 1.

First, the operation of wires in an actuation motion will be described.

Referring to FIG. 8, when the actuation lever 261 rotates in the direction of arrow OPA1 around the rotation shaft 241, the pulley 262 connected to the actuation lever 261 rotates, and the wire 301 and the wire 305 wound around the pulley 262 move in directions W1a and W1b, respectively, such that the first jaw 1101 of the end tool 1100 rotates in the direction of arrow EPA1.

Referring to FIG. 7, when the actuation lever 261 rotates in the direction of arrow OPA2 around the rotation shaft 241, the pulley 262 connected to the actuation lever 261 rotates, and both the wire 302 and the wire 306 wound around the pulley 262 move in directions W2a and W2b, respectively, such that the second jaw 1102 of the end tool 1100 rotates in the direction of arrow EPA2. Thus, when the user manipulates the actuation lever 261 toward the handle, the first jaw 1101 and the second jaw 1102 move toward each other.

Next, the operation of wires in a yaw motion will be described.

First, because the rotation shaft 243, the rotation shaft 241, and the rotation shaft 242 are connected by the yaw frame 207 (see FIG. 3), the rotation shaft 243, the rotation shaft 241, and the rotation shaft 242 rotate together.

Referring to FIG. 8, when the handle 204 rotates in the direction of arrow OPY 1 around the rotation shaft 243, the pulley 262, the pulley 211, the pulley 212, the pulley 213, and the pulley 214, and the wire 301 and the wire 305 wound therearound are rotate together around the rotation shaft 243, such that the wire 301 and the wire 305 that are wound around the pulley 213 and the pulley 214 move in the directions W1a and W1b, respectively, and thus, the first jaw 1101 of the end tool 1100 rotates in the direction of arrow EPY1.

Referring to FIG. 7, when the handle 204 rotates in the direction of arrow OPY2 around the rotation shaft 243, the pulley 262, the pulley 221, the pulley 222, the pulley 223, and the pulley 224, and the wire 302 and the wire 306 wound therearound rotate together around the rotation shaft 243, such that the wire 302 and the wire 306 that are wound around the pulley 223 and the pulley 224 move to the directions opposite to W2a and W2b, respectively, and thus, the second jaw 1102 of the end tool 1100 rotates in the direction of arrow EPY2.

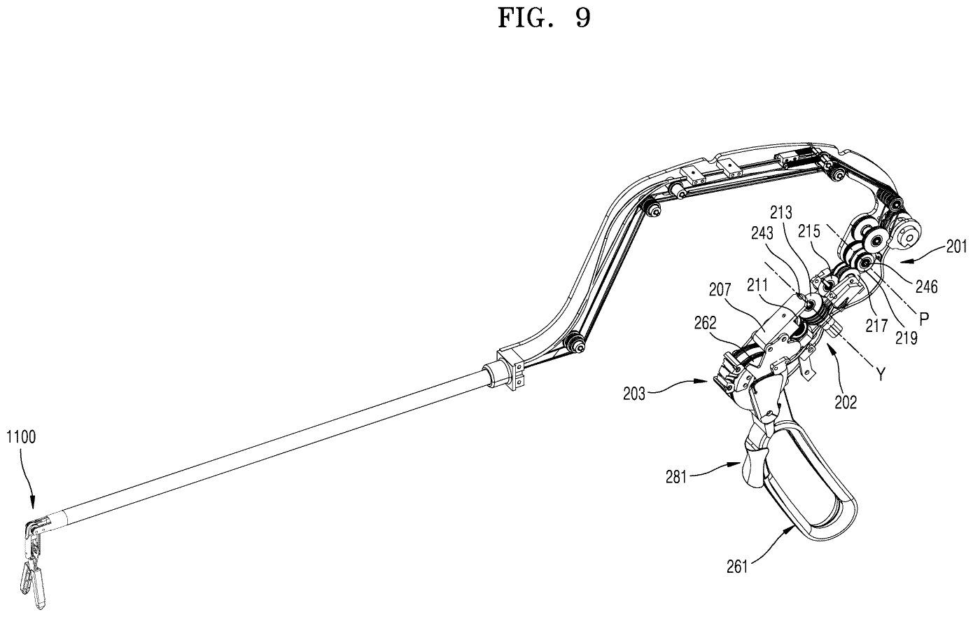

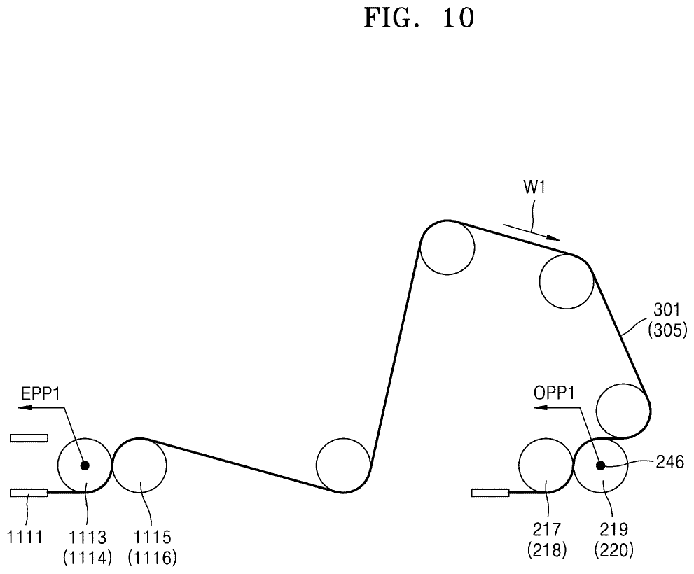

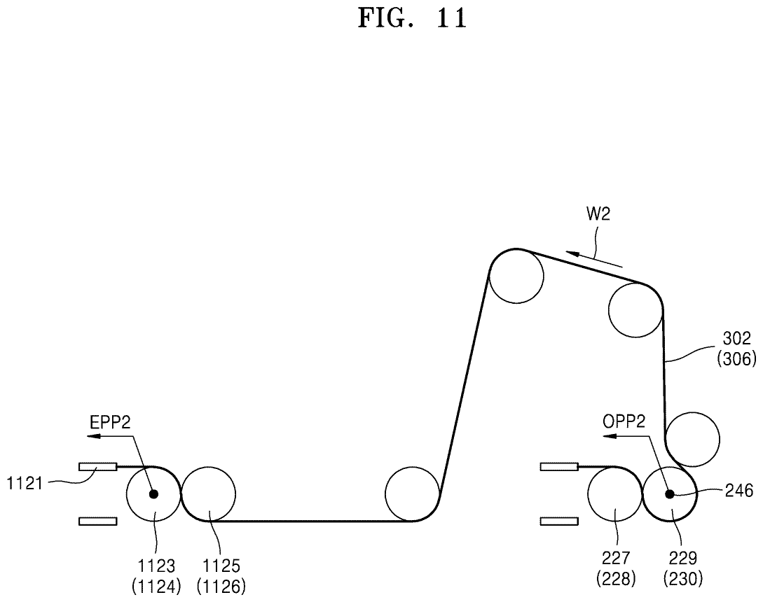

FIG. 10 and FIG. 11 are diagrams illustrating in detail a configuration of pulleys and wires associated with a pitch motion of the surgical instrument 10 for electrocautery illustrated in FIG. 1, with respect to the first jaw and the second jaw, respectively, according to an embodiment of the present disclosure. FIG. 11 is a diagram illustrating only pulleys and wires associated with the second jaw, and FIG. 10 is a diagram illustrating only pulleys and wires associated with the first jaw. As illustrated in FIG. 1, etc., there are two pulleys associated with the pitch motion, and both strands of each wire are wound along the same path, which is expressed as one line in FIG. 10. In addition, FIG. 9 is a perspective view illustrating a pitch motion of the surgical instrument of FIG. 1.

Referring to FIG. 10, when the handle 204 rotates in the direction of arrow OPP1 around the rotation shaft 246, the pulley 262, the pulley 217, and the pulley 219, and the wire 301 wound therearound rotate together around the rotation shaft 246. At this time, the wire 301 and the wire 305, which are first jaw wires, are wound on upper portions of the pulley 219 and the pulley 220, and thus move in the direction of arrow W1. Accordingly, the first jaw 1101 of the end tool 1100 rotates in the direction of arrow EPP1.

Referring to FIG. 11, when the handle 204 rotates in the direction of arrow OPP2 around the rotation shaft 246, the pulley 262, the pulley 227, and the pulley 229, and the wire 302 and the like wound therearound rotate together around the rotation shaft 246. At this time, the wire 302 and the wire 306, which are second jaw wires, are wound on lower portions of the pulley 229 and the pulley 230, and thus move in the direction of arrow W2. Accordingly, the second jaw 1102 of the end tool 1100 rotates in the direction of arrow EPP2.

Thus, actuation manipulation, yaw manipulation, and pitch manipulation may be performed independently of each other.

### Operation of Actuation Lever

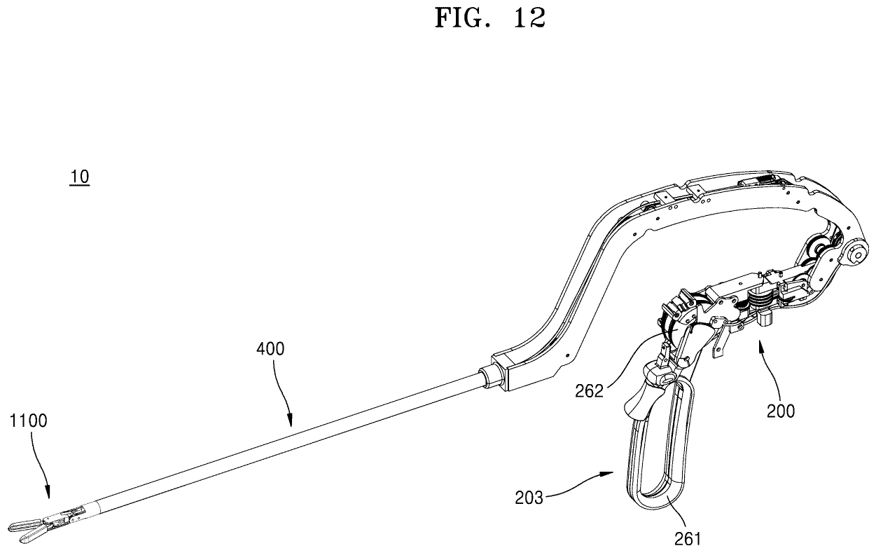

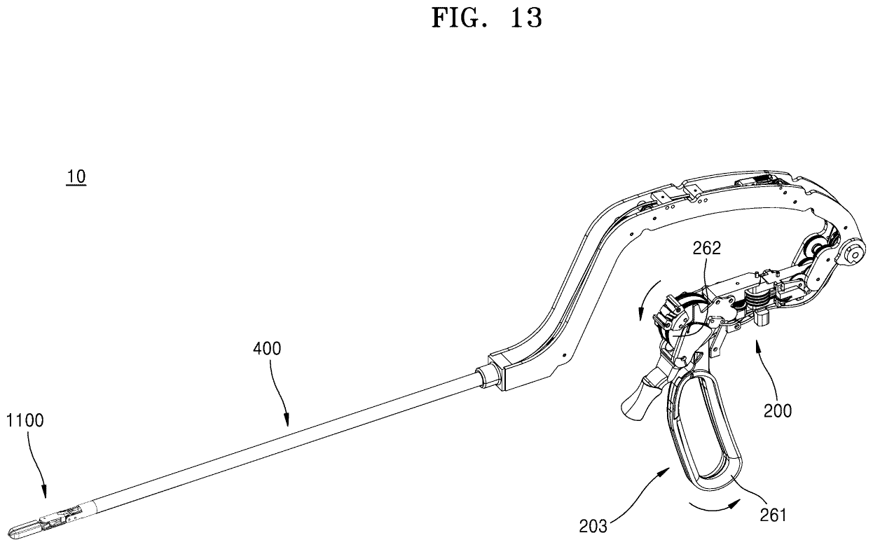

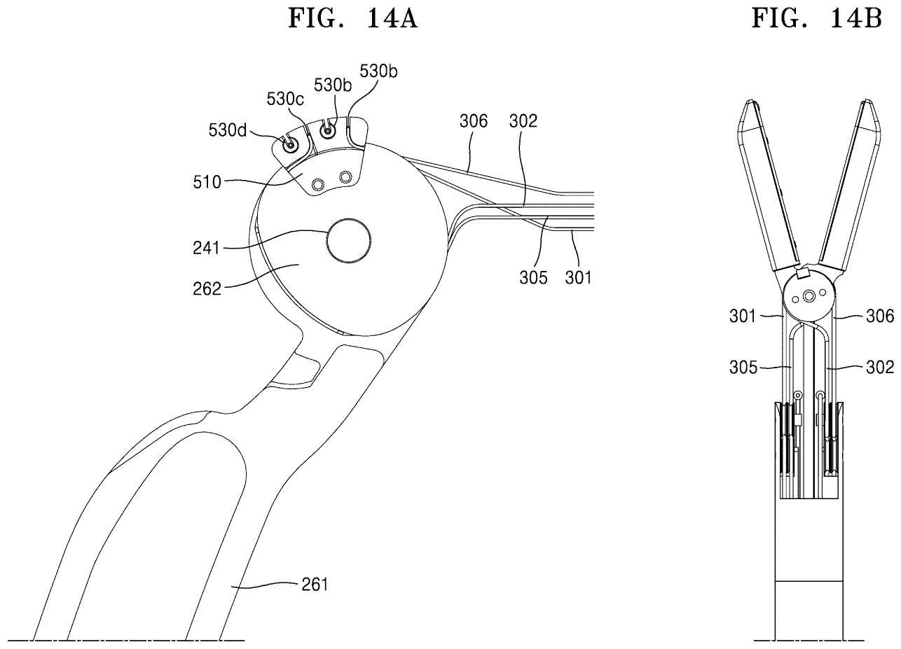

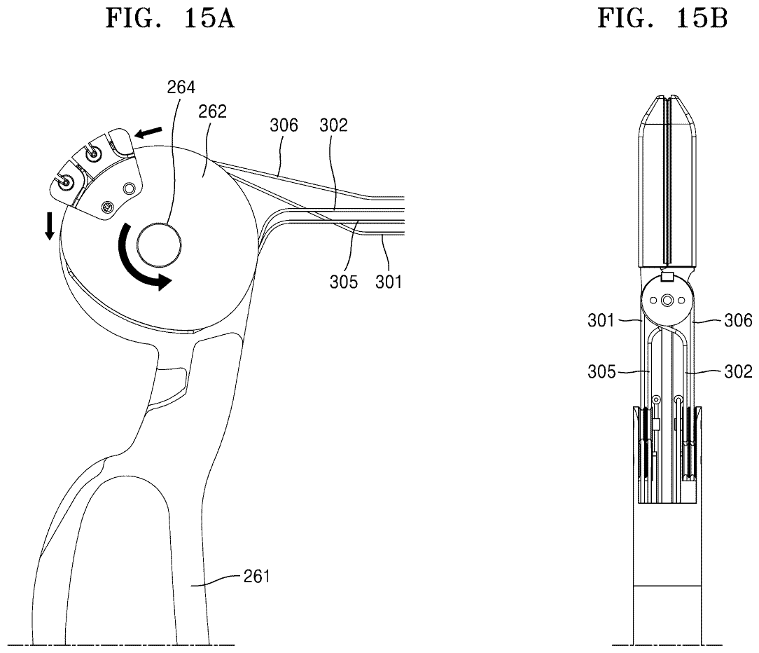

FIGS. 12 and 13 are perspective views illustrating an operation of an actuation lever of the surgical instrument 10 for electrocautery illustrated in FIG. 1, and FIGS. 14A and FIGS. 14B and FIGS. 15A and 15B are diagrams illustrating movements of wires during an operation of the actuation lever of the surgical instrument 10 for electrocautery illustrated in FIG. 1.

FIG. 12 illustrates a state in which the case of the surgical instrument 10 for electrocautery illustrated in FIG. 1 has been removed and the actuation lever 261 is not operating, and FIG. 13 illustrates a state in which the case of the surgical instrument 10 for electrocautery illustrated in FIG. 1 has been removed and the actuation lever is operating.

Referring to FIGS. 12 and 13, in the surgical instrument 10 for electrocautery according to an embodiment of the present disclosure, the actuation pulley 262 may be rotated by pulling the actuation lever 261 toward the handle 204 while gripping the handle 204 with the palm and inserting a finger into the actuation lever 261. That is, an actuation motion may be performed by manipulating one lever.

FIG. 14A is a side view illustrating wires in an actuation pulley 262 of the surgical instrument 10 for electrocautery illustrated in FIG. 1, and FIG. 14B is a plan view illustrating wires in the end tool 1100. FIG. 15A is a side view illustrating movements of wires in the actuation pulley 262 during an operation of the actuation lever, and FIG. 15B is a plan view illustrating movements of wires in the end tool 1100.

Referring to FIGS. 5, 14A, 14B, 15A and 15B, in the surgical instrument 10 according to an embodiment of the present disclosure, the wire 301 and the wire 305, which are first jaw wires, and the wire 302 and the wire 306, which are second jaw wires, are all connected to one actuation pulley 262, and thus, by appropriately configuring the arrangement of each wire, the movement of each wire may be changed with only rotation of one pulley. That is, it is possible to cause the first jaw 1101 and the second jaw 1102 to rotate in different directions through rotation of the actuation pulley 262 in any one direction by the actuation lever 261. In other words, the first jaw 1101 and the second jaw 1102 may perform an opening/closing operation to be opened or closed according to rotation of the actuation pulley 262.

In detail, the wire 301 and the wire 305, which are first jaw wires, may be wound around the actuation pulley 262 in opposite directions, respectively. For example, as illustrated in FIG. 5, the wire 301 may be wound counterclockwise and the wire 305 may be wound clockwise. Likewise, the wire 302 and the wire 306, which are second jaw wires, may be wound around the actuation pulley 262 in opposite directions, respectively. For example, as illustrated in FIG. 5, the wire 302 may be wound clockwise and the wire 306 may be wound counterclockwise.

Here, when the actuation pulley 262 rotates counterclockwise, the wire 301 is wound around the actuation pulley 262 and the wire (305) is unwound from the actuation pulley 262. Accordingly, the wire 301 is unwound from the first jaw pulley 1111 of the end tool 1100, and the wire 305 is wound around the first jaw pulley 1111, such that the end tool first jaw pulley 1111 rotates counterclockwise.

In addition, when the actuation pulley 262 rotates counterclockwise, the wire 306 is wound around the actuation pulley 262 and the wire 302 is unwound from the actuation pulley 262. Accordingly, the wire 306 is unwound from the second jaw pulley 1121 of the end tool 1100, and the wire 302 is wound around the second jaw pulley 1121, such that the end tool second jaw pulley 1121 rotates clockwise.

Likewise, when the actuation pulley 262 rotates clockwise, the first jaw pulley 1111 rotates clockwise and the second jaw pulley 1121 rotates counterclockwise.

Thus, when the actuation pulley 262 rotates, the first jaw pulley 1111 and the second jaw pulley 1121 rotate in opposite directions, causing the first jaw 1101 and the second jaw 1102 of the end tool 1100 to be open or closed.

In addition, as described above, when the actuation lever 261 rotates toward the handle 204, an actuation motion to close the end tool 1100 may be performed, and when the actuation lever 261 rotates away from the handle 204, an actuation motion to open the end tool 1100 may be performed.

In embodiments of the present disclosure, the actuation manipulation part 203 includes an elastic member 263 to enable the actuation lever 261 to automatically rotate away from the handle 204 without an additional action by the user. That is, when the user rotates the actuation lever 261 toward the handle 204 by using his/her finger, and then releases the actuation lever 261, the actuation lever 261 may automatically return to its original position by receiving a restoring force from the elastic member 263, causing the end tool 1100 to be opened.

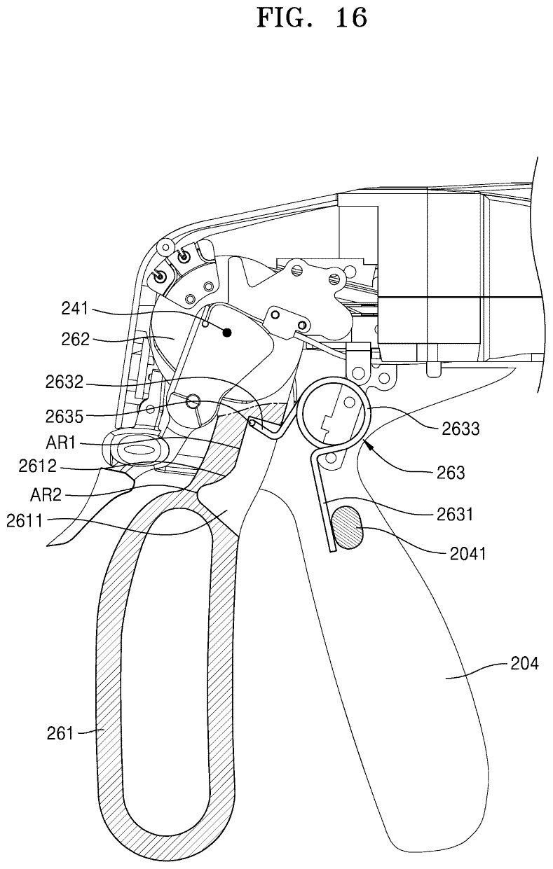

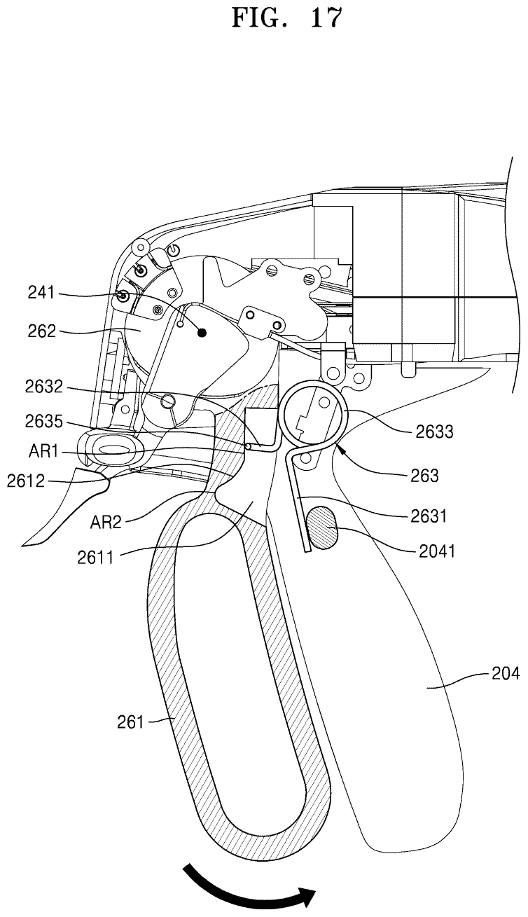

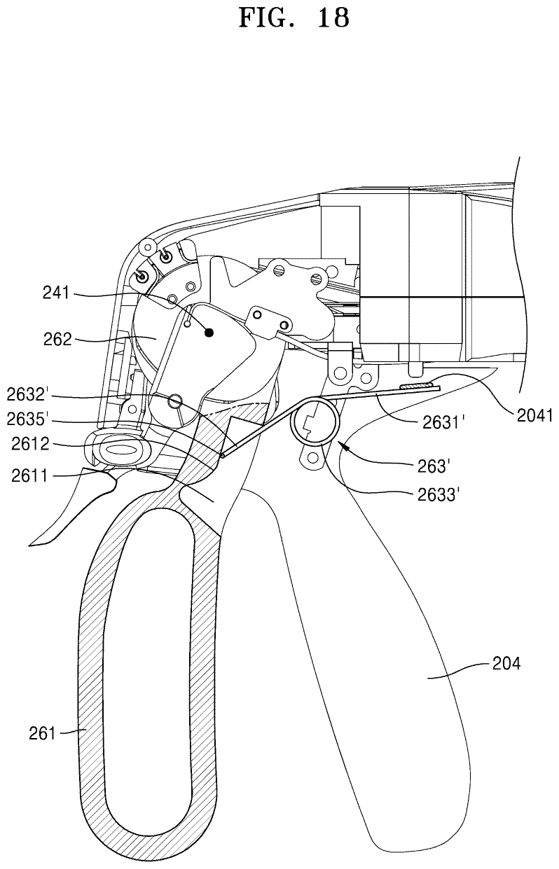

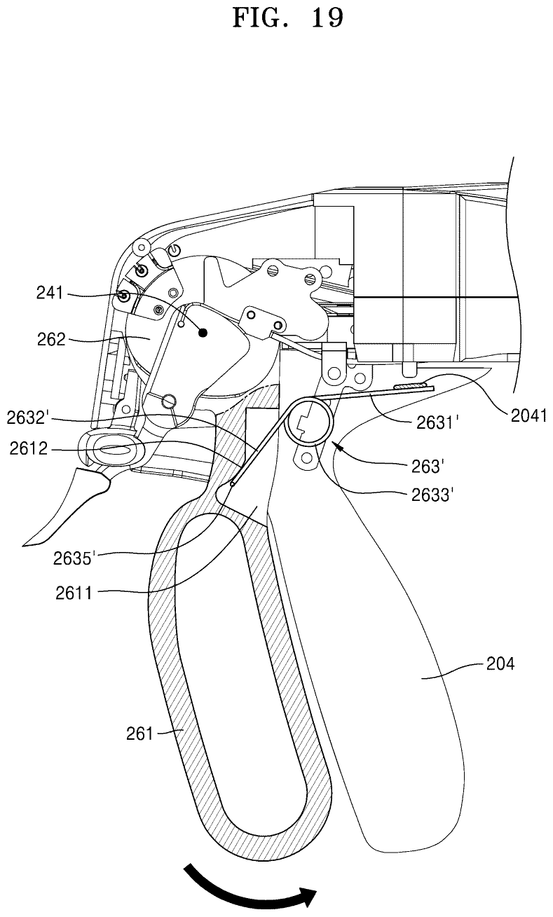

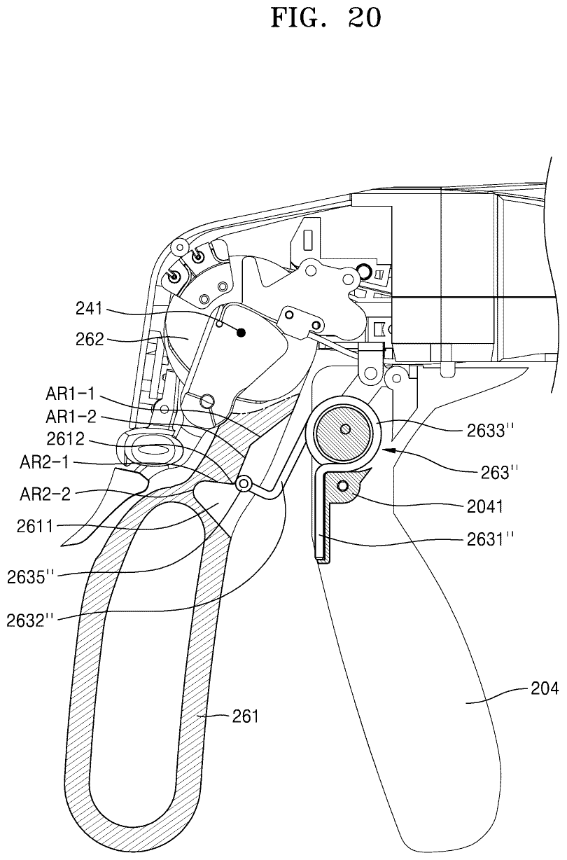

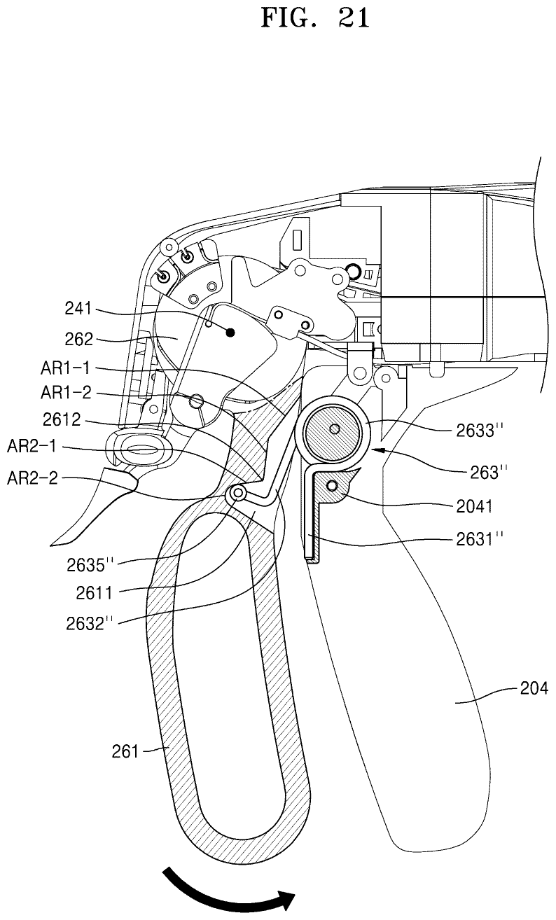

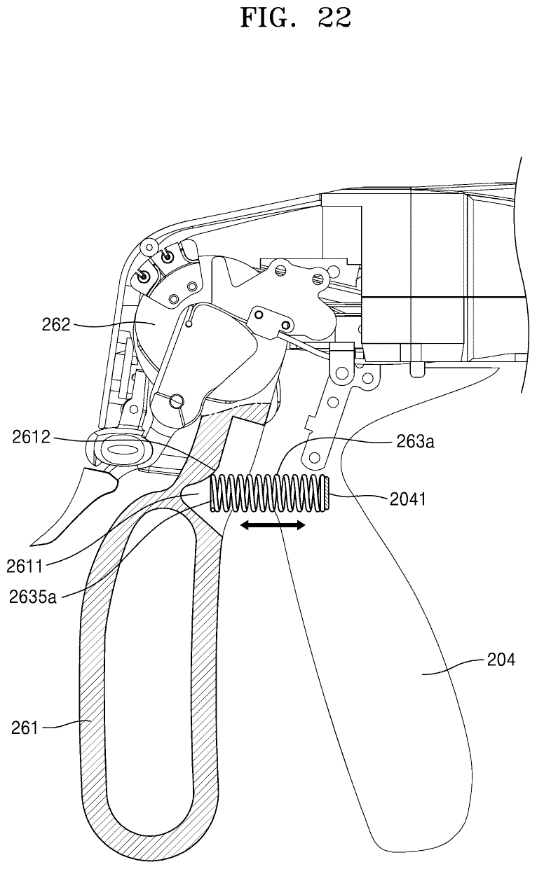

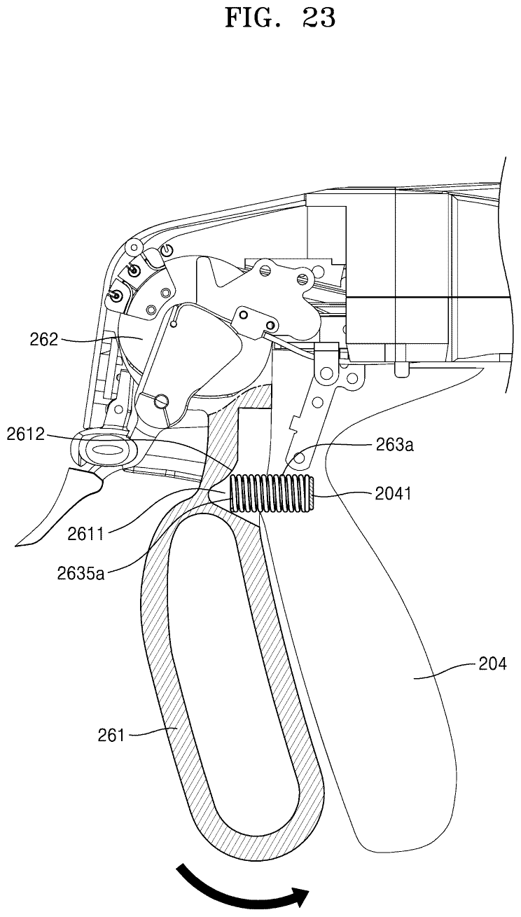

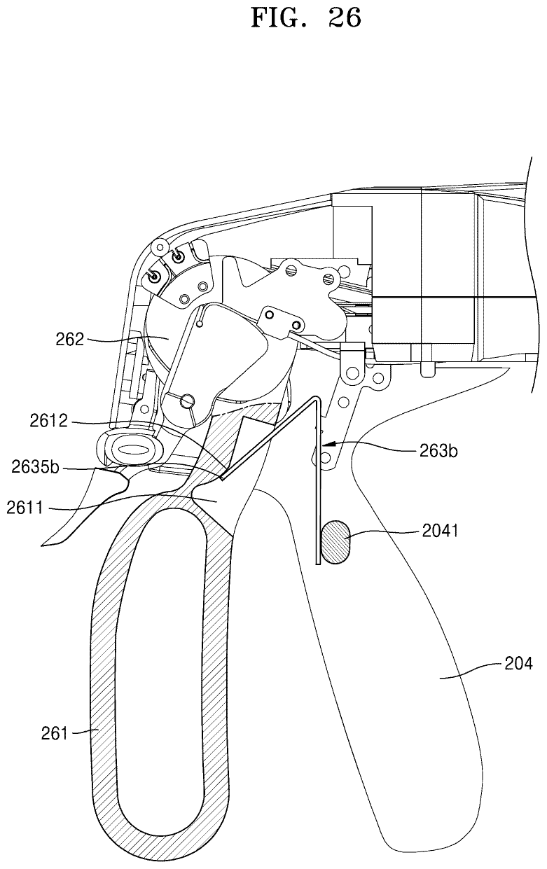

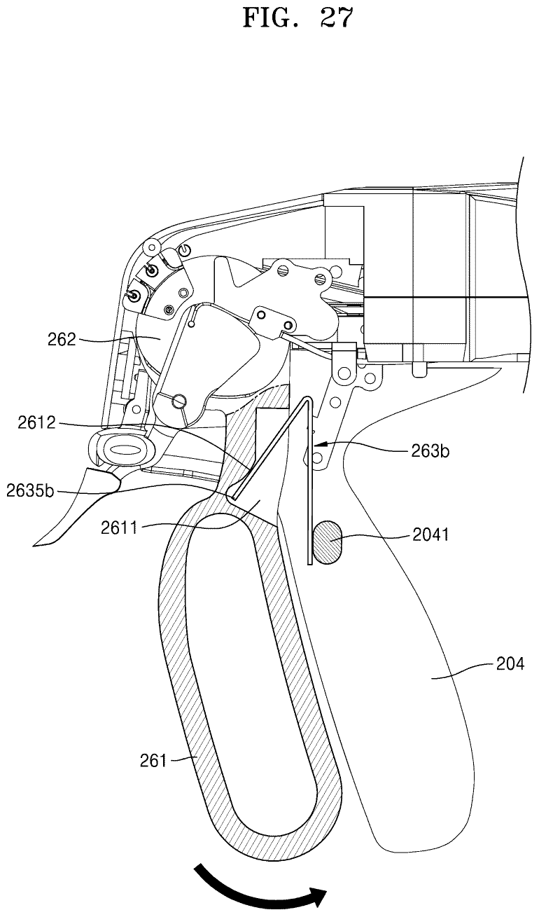

FIG. 16 is a diagram illustrating the elastic member 263 of the surgical instrument 10 for electrocautery according to an embodiment of the present disclosure, and FIG. 17 is a diagram illustrating a state in which the elastic member 263 is elastically deformed during an operation of the actuation lever of the surgical instrument 10 for electrocautery of FIG. 16. FIGS. 18 and 19 are diagrams illustrating a modified example of the elastic member 263 of FIG. 16 and FIG. 17, and FIGS. 20 and 21 are diagrams illustrating another modified example of the elastic member 263 of FIG. 16 and FIG. 17. FIGS. 22 and 23 are diagrams illustrating another embodiment of the elastic member 263 of FIGS. 16 and 17, and FIGS. 24 and 25 are diagrams illustrating a modified example of the elastic member 263a of FIGS. 22 and 23. In addition, FIGS. 26 and 27 are diagrams illustrating another embodiment of the elastic member 263 of FIGS. 16 and 17.

Referring to FIGS. 16 to 27, the actuation manipulation part 203 according to an embodiment of the present disclosure may include the elastic member 263 arranged between the handle 204 and the actuation lever 261. Here, both ends of the elastic member 263 may be arranged in the handle 204 and the actuation lever 261, respectively, to be elastically deformed between the actuation lever 261 and the handle 204 by rotation of the actuation lever 261.

The elastic member 263 may be elastically deformed when the actuation lever 261 rotates in one direction toward the handle 204, thereby providing a restoring force to the actuation lever 261 such that the actuation lever 261 rotates in another direction away from the handle 204. The user supporting the handle 204 with his/her palm may pull the actuation lever 261 toward the handle 204 by using his/her fingers, and then release the actuation lever 261. Thus, the elastic member 263 may provide a restoring force to the actuation lever 261, and accordingly, the actuation lever 261 may return to its original position.

Any configuration that may provide an elastic force between the actuation lever 261 and the handle 204 may be applied to the elastic member 263. For example, springs of various shapes and types, such as a torsion spring, a compression spring, or a plate spring, may be selected as the elastic member 263. In addition, the elastic member 263 may be one of various members formed by any one or a combination of the above springs.

The actuation manipulation part 203 according to an embodiment of the present disclosure includes the elastic member 263 to enable the actuation lever 261 to return to its original position without the user applying a separate force to the actuation lever. That is, when the user releases a grip motion of pulling the actuation lever 261, the actuation lever 261 may automatically rotate away from the handle 204 by a restoring force of the elastic member 263.

In addition, as the actuation lever 261 gets closer to the handle 204, the compressive force of the elastic member 263 increases, and thus, the user needs to apply a greater force to the actuation lever 261 when performing a grip motion of pulling the actuation lever 261 toward the handle 204.

In detail, the force required to deform the elastic member 263 is proportional to the amount of elastic deformation of the elastic member 263. In a case in which the elastic member 263 is elastically deformed at a constant rate in response to the actuation lever 261 being pulled by the user, the force required to deform the elastic member 263 gradually increases, and thus, the user needs to apply an increasingly greater force to the actuation lever 261.

Accordingly, the actuation manipulation part 203 of the present disclosure may adjust the amount of deformation of the elastic member 263 according to the rotation angle of the actuation lever 261 during a grip process in which the user pulls the actuation lever 261.

In an embodiment, the elastic member 263 of the present disclosure may have a slide portion 2635. In the present specification, the term ‘slide portion’ refers to a partial area or component of the elastic member 263 that may slide while in contact with one of the handle 204 and the actuation lever 261. That is, the ‘slide portion’ is not limited to a specific part of the elastic member 263, and may be set in various ways depending on the arrangement relationship between the elastic member 263, the handle 204, and the actuation lever 261.

One of the handle 204 and the actuation lever 261 may include a slide guide 2611. Here, the slide guide 2611 may be provided in one of the handle 204 and the actuation lever 261, which comes into contact with the slide portion 2635 of the elastic member 263 described above. The slide guide 2611 may provide a path along which the slide portion 2635 may slide, and may guide a movement of the slide portion 2635.

Hereinafter, an embodiment will be mainly described in which one end of the elastic member 263 is arranged in the actuation lever 261 and has formed therein the slide portion 2635 that may slide while in contact with the actuation lever 261, and the other end of the elastic member 263 is arranged in the handle 204, and fixed to the handle 204 by a fixing member 2041, as illustrated in FIGS. 16 to 23. Thus, the slide guide 2611 may be formed in the actuation lever 261 that comes into contact with the slide portion 2635 of the elastic member 263. In addition, in another embodiment, in a case in which one end of the elastic member 263 is fixed to the actuation lever 261, the slide portion 2635 that may slide while in contact with the handle 204 may be formed at the other end of the elastic member 263, and the slide guide 2611 may be formed in the handle 204.

In an embodiment, the slide guide 2611 may have any shape suitable for guiding the slide portion 2635 formed at one end of the elastic member 263 to move during an actuation motion. In detail, the slide guide 2611 may be formed as a recessed area or a groove that forms a certain space along one side of the actuation lever 261. The slide guide 2611 may be formed to be recessed from the outer surface of the actuation lever 261 to have a certain depth, and a portion of the elastic member 263 may be inserted into the slide guide 2611 and then move along the slide guide 2611.

Here, a contact surface 2612 that may come into contact with the slide portion 2635 may be formed on one side of the slide guide 2611. Because the slide portion 2635 of the elastic member 263 slides rather than being fixed to any one point, the point on the contact surface 2612 with which the slide portion 2635 comes into contact may change when the actuation lever 261 rotates. In other words, when the actuation lever 261 rotates toward the handle 204, the point with which the slide portion 2635 comes into contact may move away from the rotation shaft 241 on the contact surface 2612.

Thus, as the rotation angle of the actuation lever 261 increases, the amount of elastic deformation of the elastic member 263 may not increase but may remain constant or may decrease, and accordingly, the actuation manipulation part 203 may improve the stability and user convenience of a grip motion.

In an embodiment, the contact surface 2612 of the slide guide 2611 may be formed to have a certain curvature. That is, the elastic member 263 may be elastically deformed as the slide portion 2635 slides along the curvature of the contact surface 2612. This allows the user to apply a less force to the actuation lever 261 to rotate the actuation lever 261 toward the handle 204.

In a case in which the contact surface 2612 of the slide guide 2611 has a certain curvature, the amount of elastic deformation of the elastic member 263 according to rotation of the actuation lever 261 may change. In a case in which the amount of elastic deformation of the elastic member 263 remains constant or decreases when the rotation angle of the actuation lever 261 increases, the user may pull the actuation lever 261 by applying only a less force to the actuation lever 261.

For example, as illustrated in FIG. 16, the contact surface 2612 may be divided into a first area AR1 having a certain inclination, and a second area AR2 extending from the first area AR1 and having a curvature. When no external force is applied to the actuation lever 261, the slide portion 2635 of the elastic member 263 may be positioned in the first area A R 1. When the actuation lever 261 rotates in one direction toward the handle 204, the slide portion 2635 first slides along the first area AR1. When the slide portion 2635 passes through the first area AR1, the amount of deformation of the elastic member 263 according to rotation of the actuation lever 261 remains constant. Thereafter, when the slide portion 2635 slides along the second area AR2, the amount of deformation of the elastic member 263 according to rotation of the actuation lever 261 may decrease compared to when passing through the first area AR1. Thus, the user may pull the actuation lever 261 toward the handle 204 by using a less force.

In another example, as shown in FIG. 20, the first area AR1 may be formed by connecting a first-1 surface AR1-1 and a first-2 surface AR1-2 having different inclinations. In addition, the second area AR2 may be formed by connecting a second-1 surface AR2-1 having a constant inclination and a second-2 surface AR2-2 having a curvature. When the actuation lever 261 rotates in one direction toward the handle 204, the elastic member 263 is compressed, so the user has to apply a greater force to rotate the actuation lever 261. To prevent this, the second-2 surface AR2-2 is formed to have a curvature and to be recessed deepest in the actuation lever 261, thereby reducing the amount of deformation of the elastic member 263 according to the rotation of the actuation lever 261. In addition, when the grip motion of the actuation lever 261 is released, the slide part 2635 sequentially moves along the second-1 surface AR2-1, the first-2 surface AR1-2, and the first-1 surface AR1-1, which have different inclinations, so that the deformed elastic member 263 can smoothly return to its initial state.

As such, according to the present disclosure, the contact surface 2612 has a certain curvature and the slide portion 2635 of the elastic member 263 move along the curvature, such that a force applied by the user to pull the actuation lever 261 decreases or remains constant. Also, when the user releases the grip motion of pulling the actuation lever 261, the elastically deformed elastic member 263 can smoothly return to its initial state while moving along the curvature. Accordingly, the usability of the surgical instrument 10 for electrocautery of the present disclosure may be improved.

In addition, the shape of the contact surface 2612 of the slide guide 2611 is not limited thereto, and the entire contact surface 2612 may have a constant inclination or a constant curvature. That is, the shape of the contact surface 2612 may be appropriately selected to adjust the operational difficulty of the actuation lever 261 according to the purpose and user of the surgical instrument 10 for electrocautery, etc.

Hereinafter, driving of the actuation lever 261 according to the type, shape, and deformation manner of the elastic member 263 will be described.

First, as illustrated in FIGS. 16 and 17, the elastic member 263 may be an elastically deformable torsion spring.

Referring to FIGS. 16 and 17, the elastic member 263, which is a torsion spring, may include a coil portion 2633, a first spring arm 2631, and a second spring arm 2632. The coil portion 2633 may be a deformation axis of the torsion spring, the first spring arm 2631 may extend from the coil portion 2633 to be connected to the handle 204, and the second spring arm 2632 may extend from the coil portion 2633 to be connected to the actuation lever 261. Here, the slide portion 2635 may be formed in the second spring arm 2632. That is, in the elastic member 263, the coil portion 2633, the first spring arm 2631, and the second spring arm 2632 may be formed as one body, and may be elastically deformed such that the angle between the first spring arm 2631 and the second spring arm 2632 changes around the coil portion 2633 according to rotation of the actuation lever 261.

The coil portion 2633 of the elastic member 263 may be arranged to be spaced apart from the rotation shaft 242. In a case in which the rotation axis of the actuation lever 261 and the deformation axis of the torsion spring are spaced apart from each other, the amount of deformation of the torsion spring decreases when the actuation lever 261 rotates, compared to in a case in which the rotation axis and the deformation axis are arranged at the same position. Thus, the usability of the surgical instrument 10 for electrocautery may be improved by allowing the force required to rotate the actuation lever 261 to decrease or remain constant.

FIGS. 18 and 19 illustrate a modified example 263′ of the elastic member of FIGS. 16 and 17. While the first spring arm 2631 of the elastic member 263 of FIGS. 16 and 17 is fixed to an area of the handle 204 that the user holds with his/her palm, a first spring arm 2631′ of the elastic member 263′ of FIGS. 18 and 19 is fixed to an area of the handle 204 that supports pulleys. In this case, the range of variation of the angle formed by the first spring arm 2631′ and a second spring arm 2632′ around a coil portion 2633′ may increase.

FIGS. 20 and 21 illustrate another modified example of the elastic member 263″ of FIGS. 16 and 17. The elastic member 263″ of FIGS. 20 and 21 may be provided with a roller assembled at an end of the second spring arm 2632″ of the elastic member 263″, to which the slide part 2635″ is attached. Accordingly, the elastic member 263″ allows the slide part 2635″ to effectively slide along the contact surface 2612 of the slide guide 2611.

FIGS. 22 and 23 illustrate another embodiment of the elastic member 263 of FIGS. 16 and 17, in which an elastic member 263a is an elastically deformable compression spring.

Referring to FIGS. 22 and 23, when the user pulls the actuation lever 261 and rotates it in one direction toward the handle 204, the elastic member 263a, which is a compression spring, may be compressed. Thereafter, when the user no longer applies a force to the actuation lever 261, the elastic member 263a, which is a compression spring, expands to provide a restoring force to the actuation lever 261. As such, the elastic member 263a may be provided as a compressible or expandable compression spring, to apply a restoring force to cause the actuation lever 261 to rotate in another direction away from the handle 204.

As described above, the elastic member 263a, which is a compression spring, may have one end fixed by the fixing member 2041 of the handle 204, and a slide portion 2635a formed at the other end of the elastic member 263a may slide while in contact with the slide guide 2611 of the actuation lever 261. That is, the elastic member 263a slides along the contact surface 2612 of the slide guide 2611 to be compressed or expand. Here, in a case in which the contact surface 2612 has a certain curvature, the amount of compression of the elastic member 263a according to the degree of rotation of the actuation lever 261 may change such that the force necessary for the user to rotate the actuation lever 261 decreases.

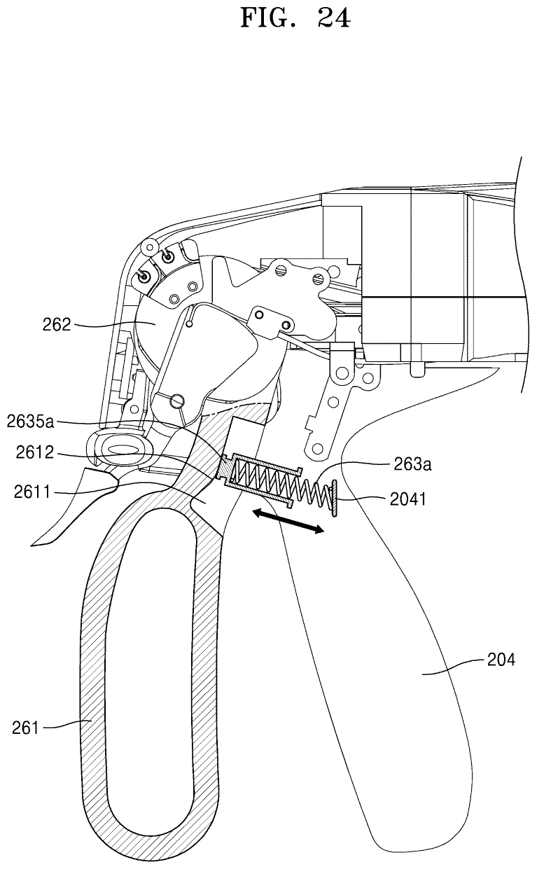

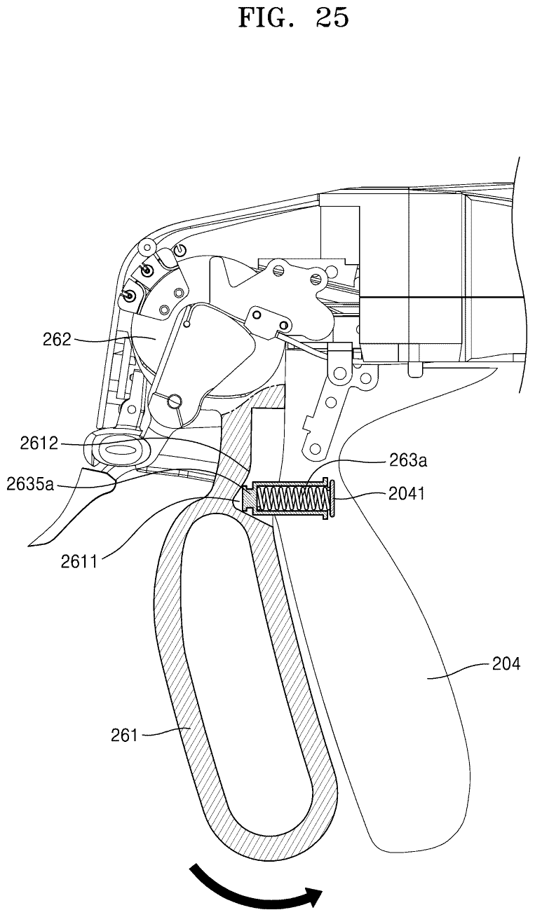

FIGS. 24 and 25 illustrate a modified example of the elastic member 263a of FIGS. 22 and 23. The slide portion 2635a of the elastic member 263a, which is a compression spring, may have parts of various shapes and materials suitable for sliding along the slide guide 2611. For example, the slide portion 2635a may include a cap coupled to the other end of the elastic member 263a as illustrated in FIGS. 24 and 25. Alternatively, the slide portion 2635a may have a contact protrusion (not shown) protruding from the other end of the elastic member 263a. Accordingly, the elastic member 263a, which is a compression spring, may slide while minimizing friction with the slide guide 2611.

FIGS. 26 and 27 illustrate another embodiment of the elastic member 263 of FIGS. 16 and 17, in which an elastic member 263b is a plate spring.

Referring to FIGS. 26 and 27, the elastic member 263b, which is a plate spring, may be a plate-shaped spring that is bent to have a certain included angle, with both ends arranged in the handle 204 and the actuation lever 261, respectively. When the user pulls the actuation lever 261 and rotates the actuation lever 261 in one direction toward the handle 204, the elastic member 263b, which is a plate spring, may be elastically deformed such that the included angle decreases. Thereafter, when the user no longer applies a force to the actuation lever 261, the elastic member 263b, which is a plate spring, may be elastically deformed to increase the included angle, and provide a restoring force to the actuation lever 261. As such, the elastic member 263b is provided as a spring in the shape of a certain bent plate, to apply a restoring force to cause the actuation lever 261 to rotate in another direction away from the handle 204.

As described above, the elastic member 263b, which is a plate spring, may have one end fixed by the fixing member 2041 of the handle 204, and a slide portion 2635b formed at the other end of the elastic member 263b may slide while in contact with the slide guide 2611 of the actuation lever 261. That is, the elastic member 263b may be elastically deformed to slide along the contact surface 2612 of the slide guide 2611 to change the included angle. Here, in a case in which the contact surface 2612 has a certain curvature, the amount of deformation of the elastic member 263b, that is, the amount of change in the included angle, may decrease according to the degree of rotation of the actuation lever 261, such that the force necessary for the user to rotate the actuation lever 261 decreases.

As such, the elastic member 263 of the present disclosure may be one of various elastically deformable members, and may be appropriately selected in consideration of the elastic deformation manner of each member, the purpose and user of the surgical instrument 10 for electrocautery, etc.

The surgical instrument for electrocautery according to an embodiment of the present disclosure includes an elastic member capable of providing a restoring force to the actuation lever, such that the actuation lever may automatically move away from the handle by the elastic member without a separate force applied by the user. This may improve the stability and usability of the manipulation part during an actuation motion of the end tool.

The present disclosure has been described with reference to the preferred embodiments. It will be understood by those of skill in the art that the present disclosure may be implemented in a modified form without departing from the intrinsic characteristics of the present disclosure. Therefore, the disclosed embodiments are to be considered in a descriptive sense only, and not for purposes of limitation. The scope of the present disclosure is in the claims rather than the above descriptions, and all differences within the equivalent scope should be construed as being included in the present disclosure.

In a surgical instrument for electrocautery according to the present disclosure, an actuation lever automatically returns to its initial position by a restoring force provided by an elastic member, even when a user does not apply a separate force, thereby improving the convenience of an operator and the safety and usability of the surgical instrument.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A manipulation part for a surgical instrument comprising an end tool, the manipulation part comprising:
a handle that is grippable; and
an actuation manipulation part formed on one side of the handle and configured to control an actuation motion of the end tool,
wherein the actuation manipulation part comprises:
an actuation lever that is rotatable around an actuation rotation shaft; and
an elastic member that is arranged between the handle and the actuation lever to be elastically deformable, and provides a restoring force to the actuation lever, and
the elastic member comprises a slide portion that is slidable while in contact with one of the handle and the actuation lever.

2. The manipulation part of claim 1, wherein, in response to the actuation lever rotating in one direction, the elastic member is elastically deformed to provide the restoring force to cause the actuation lever to rotate in another direction.

3. The manipulation part of claim 1, wherein one of the handle and the actuation lever, which comes into contact with the elastic member, comprises a slide guide that guides sliding of the slide portion of the elastic member.

4. The manipulation part of claim 3, wherein the slide guide is formed to be recessed from one side of one of the handle and the actuation lever, which comes into contact with the elastic member, and
a portion of the elastic member is inserted into the slide guide to be movable along the slide guide.

5. The manipulation part of claim 3, wherein, in the slide guide, at least a portion of a contact surface that comes into contact with the slide portion is formed to have a certain curvature.

6. The manipulation part of claim 5, wherein the contact surface comprises a first area having a constant inclination, and a second area extending from the first area and having a curvature.

7. The manipulation part of claim 6, wherein the slide portion of the elastic member slides along the first area and the second area, to reduce an amount of elastic deformation according to rotation of the actuation lever.

8. The manipulation part of claim 1, wherein the elastic member is a compression spring having one end slidably supported by one of the handle and the actuation lever, and another end fixed to the other one of the handle and the actuation lever.

9. The manipulation part of claim 1, wherein the elastic member is a torsion spring comprising a first spring arm extending from a coil portion to be connected to the handle, and a second spring arm extending from the coil portion to be connected to the actuation lever.

10. The manipulation part of claim 9, wherein the coil portion is arranged to be spaced apart from the actuation rotation shaft.

11. A surgical instrument for electrocautery comprising:
an end tool that is rotatable in at least one direction;
a manipulation part comprising a handle that is grippable, and an actuation manipulation part that is formed on one side of the handle and configured to control an actuation motion of the end tool; and
a power transmission part that comprises an actuation wire connecting the end tool to the actuation manipulation part, and is configured to transmit power from the actuation manipulation part to the end tool,
wherein the actuation manipulation part comprises:
an actuation lever that is rotatable around an actuation rotation shaft; and
an elastic member that is arranged between the handle and the actuation lever to be elastically deformable, and provides a restoring force to the actuation lever to offset tension of the actuation wire.

12. The surgical instrument for electrocautery of claim 11, wherein, in response to the actuation lever rotating in one direction, the elastic member is elastically deformed to provide the restoring force to cause the actuation lever to rotate in another direction.

13. The surgical instrument for electrocautery of claim 11, wherein the elastic member comprises a slide portion that is slidable while in contact with one of the handle and the actuation lever.

14. The surgical instrument for electrocautery of claim 13, wherein one of the handle and the actuation lever, which comes into contact with the elastic member, comprises a slide guide that comes into contact with the slide portion of the elastic member to guide sliding of the slide portion.

15. The surgical instrument for electrocautery of claim 14, wherein the slide guide is formed to be recessed from one side of one of the handle and the actuation lever, which comes into contact with the elastic member, and
a portion of the elastic member is inserted into the slide guide to be movable along the slide guide.

16. The surgical instrument for electrocautery of claim 14, wherein, in the slide guide, at least a portion of a contact surface that comes into contact with the slide portion is formed to have a certain curvature.

17. The surgical instrument for electrocautery of claim 16, wherein the contact surface comprises a first area having a constant inclination, and a second area extending from the first area and having a curvature.

18. The surgical instrument for electrocautery of claim 17, wherein the slide portion of the elastic member slides along the first area and the second area, to reduce an amount of elastic deformation according to rotation of the actuation lever.

19. The surgical instrument for electrocautery of claim 11, wherein the elastic member is a compression spring having one end slidably supported by one of the handle and the actuation lever, and another end fixed to the other one of the handle and the actuation lever.

20. The surgical instrument for electrocautery of claim 11, wherein the elastic member is a torsion spring comprising a first spring arm extending from a coil portion to be connected to the handle, and a second spring arm extending from the coil portion to be connected to the actuation lever.
